(19) United States
(12) Patent Application Publication
(10) Pub. No.: US 2011/0039873 A1
(43) Pub. Date: Feb. 17, 2011

(54) SUBSTITUTED PYRAZOLO[1,5-a] PYRIDINE COMPOUNDS HAVING MULTI-TARGET ACTIVITY

(76) Inventors: Federico C.A. GAETA, Mountain View, CA (US); Kirk W. JOHNSON, Moraga, CA (US); Matthew I. GROSS, Vallejo, CA (US); Annemarie LEDEBOER, Alameda, CA (US)

Correspondence Address: FOLEY AND LARDNER LLP;SUITE 500, 3000 K STREET NW, WASHINGTON, DC, 20007, US

(21) Appl. No.: 12/796,164

(22) Filed: Jun. 8, 2010

Related U.S. Application Data
(60) Provisional application No. 61/185,074, filed on Jun. 8, 2009.

(51) Int. Cl. A61K 31/506; C07D 471/04; A61K 31/444; A61P 25/04

(52) U.S. Cl. 514/275; 544/331; 546/121; 514/300

(57) ABSTRACT

The substituted pyrazolo[1,5-a]pyridine compounds in accordance with Formula 1 are strong inhibitors of phosphodiesterase and c-Jun N-terminal kinase activity.

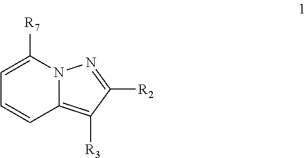

Accordingly, Formula 1 compounds are candidate therapeutics for treating disease states such as cancer, neuropathic pain, inflammation as well as cognitive disorders such as Parkinson's Disease.

This application claims the benefit of priority of U.S. Provisional Application No. 61/185,074 filed on Jun. 8, 2009. The entire disclosure of which is incorporated herein by reference.

### FIELD

This disclosure relates generally to substituted pyrazolo[1,5-a]pyridine compounds having multi-target activity. In particular, the disclosure is directed to, among other features, substituted pyrazolo[1,5-a]pyridine compounds exhibiting both phosphodiesterase (PDE) and c-Jun N-terminal kinase (JNK) inhibitory activity. The subject compounds are expected to be anti-inflammatory in general, and specifically attenuating of glial activation. Also provided is a related method for inhibiting both PDE and JNK by administering a therapeutically effective amount of a substituted pyrazolo[1,5-a]pyridine compound, to thereby treat any of a number of related disorders or conditions.

### BACKGROUND

The cyclic nucleotide phosphodiesterases (PDE) comprise a group of enzymes that degrade the phosphodiester bond in the second messenger molecules cAMP and cGMP. They regulate the localization, duration, and amplitude of cyclic nucleotide signaling within subcellular domains, and are therefore important regulators of signal transduction. The PDE superfamily currently includes more than twenty different genes subgrouped into eleven PDE families (Lugnier, C., Pharmacol Ther. 2006, 109(3):366-98). The phosphodiesterases have different substrate specificities; some are cAMP selective hydrolases (PDE4, 7 and 8), while others such as PDE5, 6, and 9, are cGMP selective, and yet other phosphodiesterases such as PDE1, 2, 3, 10, and 11, can hydrolyse both cAMP and cGMP. PDE inhibitors are compounds that block the enzyme, PDE, thereby preventing the inactivation of the intracellular second messengers cAMP and cGMP. Thus, PDE inhibitors can prolong or enhance the effects of physiological processes mediated by cAMP or cGMP. Indeed, certain PDE inhibitors have been identified as new potential therapeutics in areas such as pulmonary arterial hypertension, coronary heart disease, dementia, depression, and schizophrenia.

PDE4 is the major cAMP-metabolizing enzyme found in inflammatory and immune cells. PDE4 inhibitors have potential as anti-inflammatory drugs, especially in inflammatory pulmonary diseases such as asthma, COPD, and rhinitis. They suppress the release of cytokines and other inflammatory signals and inhibit the production of reactive oxygen species. PDE4 inhibitors may have antidepressive effects (Bobon D, et al., Eur Arch Psychiatry Neurol Sci. 1988, 238 (1), 2-6) and have also recently been proposed for use as antipsychotics (Maxwell C R, et al., 2004, 129 (1): 101-7).

PDE10 contains two amino-terminal domains that are similar to the cGMP-binding domains of PDE2, PDE5 and PDE6, which are domains conserved across a wide variety of proteins. Inhibitors of the PDE family of enzymes have widely been sought for a broad indication of therapeutic uses including allergies, obtrusive lung disease, hypertension, renal carcinoma, angina, congestive heart failure, depression and the like. Inhibitors of PDE10 have also been described for treatment of certain neurological and psychiatric disorders including Parkinson's disease, Huntington's disease, schizophrenia, delusional disorders, drug-induced psychosis and panic and obsessive-compulsive disorders (U.S. Patent Application No. 2003/0032579). PDE10 has been shown to be present at high levels in neurons in areas of the brain that are closely associated with many neurological and psychiatric disorders. By inhibiting PDE10 activity, levels of cAMP and cGMP are increased within neurons, and the ability of these neurons to function properly is thereby improved. Thus, inhibition of PDE10 may be useful in the treatment of a wide variety of conditions or disorders that would benefit from increasing levels of cAMP and cGMP within neurons, including those neurological, psychotic, anxiety and/or movement disorders mentioned above.

Jun N-terminal kinase (JNK) is a stress-activated protein kinase that can be induced by inflammatory cytokines, bacterial endotoxin, osmotic shock, UV radiation, and hypoxia. Specifically, c-Jun N-terminal kinase (JNK) is a serine threonine protein kinase that phosphorylates c-Jun, a component of the transcription factor activator protein-1. In complex with other DNA binding proteins, AP-1 regulates the transcription of numerous genes including cytokines (e.g., IFN-γ, IL-2, and tumor necrosis factor (TNF)-α;), growth factors (e.g., vascular endothelial growth factor (VEGF)), immunoglobulins (e.g., K light chain), inflammatory enzymes (e.g., COX-2), and matrix metalloproteinases (e.g., MMP-13).

JNK is a member of the mitogen-activated protein kinase (MAPK) family that includes the extracellular regulated kinases (ERKs) and p38 kinases. Three JNK genes (JNK1, -2, and -3) have been identified in humans; however, splice variants result in a total of 10 isoforms. JNK1 and JNK2 have a broad tissue distribution, whereas JNK3 seems primarily localized to neuronal tissues and the cardiac myocyte. Mice lacking JNK1 or JNK2 exhibit deficits in T-helper (CD4⁺) cell function. Double knockout animals are embryonic lethal, although fibroblasts from these animals are viable in vitro and exhibit a remarkable resistance to radiation-induced apoptosis. The JNK3 knockout mouse exhibits resistance to kainic acid-induced apoptosis in the hippocampus and to subsequent seizures. Therefore, JNK activity seems critical for both the immune response and for programmed cell death. Therapeutic inhibition of JNK may provide clinical benefit in diseases as diverse as arthritis, inflammatory bowel disease, chronic obstructive pulmonary disease, graft vs. host disease, stroke, Parkinson's disease, ischemic injury, and myocardial infarction (Bennett, B., et al., PNAS, 2001, vol. 98 no. 24 13681-13686 and references therein). Neurological conditions are additionally indicated including neurodenerative syndromes (e.g. Alzheimer's, Parkinson's) and both inflammatory and chronic neuropathic pain conditions.

### SUMMARY

The present disclosure is directed to substituted pyrazolo[1,5-a]pyridine compounds that comport with Formula I and have multi-target activity. More particularly, certain of the compounds provided herein demonstrate activity against both phosphodiesterases as well as against c-Jun N-terminal kinases (JNKs). Such multi-target activity is unique, and suggests that the subject compounds may be useful in multiple indications to be described in greater detail herein.

Each of the following embodiments described may be considered singly, or taken in combination with any one or more additional embodiments, so long as the particular combination is not mutually inconsistent with the particular embodiments included in such combination.

In a first aspect, the present disclosure provides substituted pyrazolo[1,5-a]pyridine compounds. The compounds possess a substituent at the 3-ring position, and may also possess a substituent at the 2-ring and/or 7-ring position. The compounds may generally be described as having a structure according to Formula I:

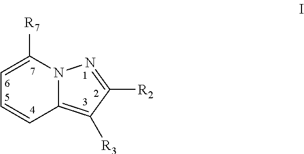

where R₃ is an amino-substituted pyrimidine or pyridine; R₂ is independently H or an organic radical selected from the group consisting of alkyl, cycloalkyl, alkoxyalkyl (e.g., compound 1117, methoxymethyl), aryl (e.g., phenyl), and haloaryl; and R₇ is independently selected from H or alkyl.

In particular embodiments, a pyrazolo[1,5-a]pyridine compound as provided herein is selected from a 2,3 substituted pyrazolo[1,5-a]pyridine compound, a 3-substituted pyrazolo[1,5-a]pyridine compound, a 3,7-substituted pyrazolo[1,5-a]pyridine compound, and a 2,3,7-substituted pyrazolo[1,5-a]pyridine compound, where the substituents at each ring position are as described herein.

In reference to the general structure I, turning now to the R₃ substituent, in a particular embodiment, R₃ is a pyrimidine possessing an amine substituent at its 2-ring position (i.e., at the carbon interposed between the two ring nitrogens of the pyrimidine) or R₃ is a pyrimidine possessing an amine substituent at its 2-ring position. In certain embodiments, when the 3-substituent of the pyrazolo[1,5-a]pyridine is a substituted pyrimidin-2-amine moiety, the pyrimidine is attached to the core system via the 4-position of the pyrimidine. (See exemplary structure below, where R₁₀ and R₁₁ are each independently selected from H, alkyl, cycloalkyl, and aliphatic 3, 4, 5, and 6-membered nitrogen containing heterocycles).

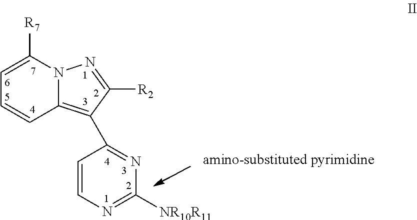

In yet another particular embodiment, R₃ is a pyridine ring possessing an amine substituent at its 2-ring position.

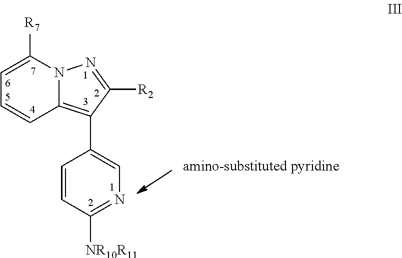

In one embodiment, R₃ is an amino-substituted pyridine as illustrated in structure IV.

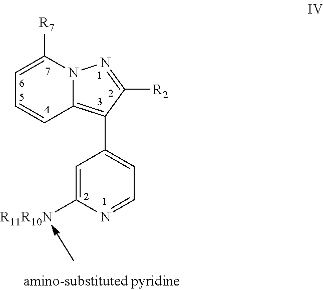

Illustrative of the amino substituted pyridine and pyrimidine are those compounds in which R₁₀ and R₁₁ are each independently selected from H, alkyl, substituted alkyl, cycloalkyl, S(O)₂R′ and aliphatic 3, 4, 5, and 6-membered nitrogen containing heterocycles. When R₁₀ or R₁₁ is S(O)₂R′, R′ is selected from the group consisting alkyl, aryl and heteroaryl. For example, R′ can be methyl, ethyl, propyl, phenyl, thiophene or quinoline.

One particular amine substituent in either structure II, III, or IV is one where R₁₀ is hydrogen.

In yet another embodiment directed to R₃, the amine substituent in either structure II, III or IV is one where R₁₀ is hydrogen and R₁₁ is lower alkyl or lower cycloalkyl. Exemplary R₁₁ substituents include methyl, ethyl, propyl, isopropyl, butyl, 2-methylpropyl, pentyl, N-3-pentyl, 1-methylbutyl, 1-ethylpropyl, 3-methylpentyl, cyclopropyl, cyclobutyl, cyclopentyl, and the like.

In yet another embodiment directed to R₃, the amine substituent in either structure II, III or IV is one where R₁₁ is an aliphatic 3, 4, 5, and 6-membered nitrogen containing heterocycle selected from aziridine, pyrrolidine, and piperidine. In a particular embodiment, R₁₁ is a pyrrolidine connected to the amine nitrogen at the 3-ring position of the pyrrolidine ring.

Illustrative amino substituents corresponding to either structure II, III or IV include the following, where the squiggly line indicates attachment to the corresponding pyrimidine or pyridine:

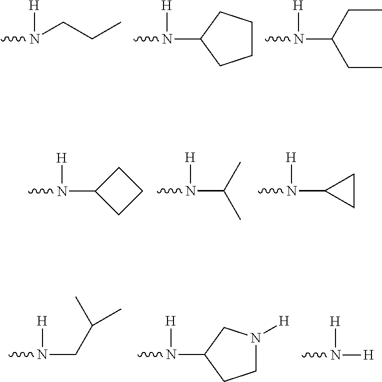

When R₃ is an amino substituted pyrimidine,

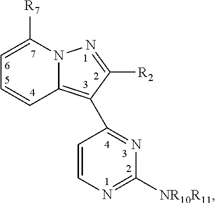

NR₁₀R₁₁ is selected from:

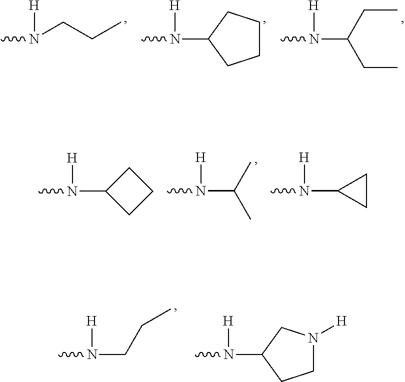

and R₂ and R₇ are as described generally above.

Additional R₃ substituents are shown in the following structures, where R₂ and R₇ are as described generally above:

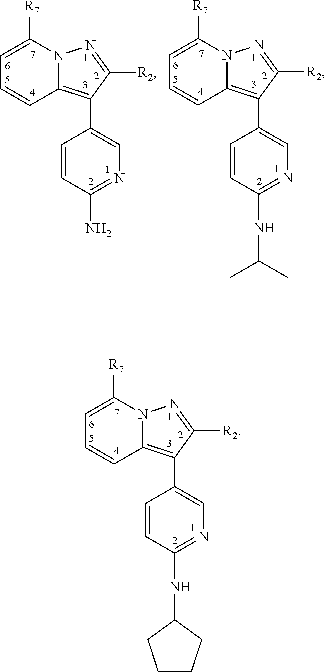

Turning now to substituent R₇, in one embodiment, R₇ is either hydrogen or lower alkyl, e.g., is selected from methyl, ethyl, propyl, isopropyl, 1-ethylpropyl, 1,2-dimethylpropyl, n-butyl, i-butyl, sec-butyl, t-butyl, and the like. In one particular embodiment, R₇ is methyl.

Turning now to R₂, as described above, R₂ typically is independently H or an organic radical selected from the group consisting of alkyl, cycloalkyl, alkoxyalkyl (e.g., compound 1117, methoxymethyl), aryl (e.g., phenyl), and haloaryl.

In one embodiment, R₂ is lower alkyl or lower cycloalkyl. Illustrative lower alkyl R₂ groups include methyl, ethyl, propyl, isopropyl, 1-ethylpropyl, 1,2-dimethylpropyl, n-butyl, i-butyl, sec-butyl, t-butyl, and the like. Lower cycloalkyl groups are selected from cyclopropyl, cyclobutyl, and cyclopentyl.

In yet another embodiment, R₂ is phenyl or is a halo-substituted phenyl. The halo substituted phenyl is selected from a phenyl ring having a single halogen substituent selected from fluorine, chlorine or bromine or iodine. In one embodiment, the halogen is chlorine or fluorine. The halogen may be at any position on the phenyl ring, e.g., alpha, meta, or para to the parent pyrazolo[1,5-a]pyridine core structure. In one particular embodiment, the halogen is at the 3-position of the phenyl ring (assuming that the 1-position of the phenyl is the attachment to the core).

In yet another embodiment, R₂ is an alkyl alkoxy group, preferably a lower alkyl lower alkoxy group/Illustrative R₂ substituents falling into this classification include methyl methoxy (—CH₂OCH₃), ethyl methoxy (—CH₂CH₂OCH₃), and the like. For instance, a lower alkyl lower alkoxy substituent may be described as —R₁₂—O—R₁₃, where R₁₂ and R₁₃ are each selected from lower alkyl, and R₁₂ is attached to the parent pyrazolo[1,5-a]pyridine core structure. An R₁₂ group may be a linear lower alkyl such as methyl, ethyl, propyl, butyl, pentyl, or hexyl, while R₁₃ taken together with the adjacent oxygen may be linear or branched alkoxy. Illustrative R₁₃ groups include methyl, ethyl, propyl, isopropyl, 1-ethylpropyl, 1,2-dimethylpropyl, n-butyl, butyl, sec-butyl, t-butyl, and the like.

In one embodiment, R₂ is selected from hydrogen, methyl, isopropyl, tert-butyl, cyclopropyl, butyl, methyl methoxy, phenyl, sec-butyl, 3-fluorophenyl, and 3-chorophenyl.

Compounds provided herein are meant to encompass the parent pyrazolo[1,5-a]pyridine core structure substituted with any combination of R₂, R₃, and R₇ moieties as provided herein, as consistent with the general features described.

In a particular embodiment, in reference to structure I, if the compound is a 2,3-substituted pyrazolo[1,5-a]pyridine and R₂ is isopropyl, then when R₃ is a substituted or unsubstituted (referring to the amine moiety) pyrimidin-2-amine moiety substituted at the 4-position of the pyrimidine ring to the pyrazolo[1,5-a]pyridine, R₃ is other than isopropylpyrimidin-2-amine (1137), pyrimidin-2-amine (1139), (pyrimidin-2-ylamino)propan-1-ol (1134) and 3-(piperazin-1-yl)propyl)pyrimidin-2-amine (1135). For example, in this embodiment, R₃ is other than

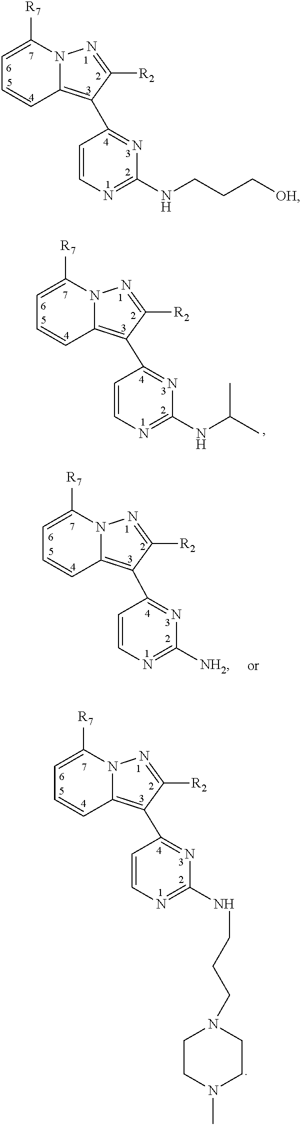

Illustrative compounds having values for R₂, R₃ and R₇ as described above are provided in Table I.

In one embodiment, a substituted pyrazolo[1,5-a]pyridine compound as provided herein is capable of inhibiting either JNK-2 or JNK-3 enzyme. Particularly preferred are substituted pyrazolo[1,5-a]pyridine compounds having an IC₅₀ value based upon a JNK inhibition assay as described herein of less than about 5.00 μM.

In a particular embodiment, the compound will possess an IC₅₀ value based upon a JNK 3 inhibition assay as described herein ranging from about 0.01 to 5.00 μM, preferably from 0.01 to 4.00 μM or more preferably from about 0.01 to about 3.00 μM. Particularly preferred are compounds having an IC₅₀ value based upon a JNK 3 inhibition assay in a range from 0.01 to 2.00 μM. Illustrative compounds particularly effective in JNK-3 inhibition include 1136, 1158, 1164, 1165, 1166, 1167, 1173, 1174, 1175, 1176, 1177, 1179, 1180, 1182, 1183, 1184, 1194, 1195, 1198, and 1200.

In yet another embodiment, the compound will possess an IC₅₀ value based upon a JNK 2 inhibition assay as described herein ranging from about 0.01 to 5.00 μM, preferably from 0.01 to 3.00 μM or more preferably from about 0.01 to about 2.00 μM. Particularly preferred are compounds having an IC₅₀ value based upon a JNK 2 inhibition assay in a range from 0.01 to 2.00 μM. Illustrative compounds particularly effective in JNK-2 inhibition and falling within this classification inhibition include 1153, 1156, 1164, 1165, 1166, 1167, 1173, 1174, 1176, 1194, 1195, 1198, and 1200.

In yet another embodiment, the substituted pyrazolo[1,5-a]pyridine compound will possess IC₅₀ values in both JNK 2 and JNK 3 inhibition assays as described herein ranging from about 0.01 to 2.00 μM. Exemplary compounds exhibiting the foregoing feature include 1137, 1164, 1165, 1166, 1167, 1173, 1174, 1176, 1177, 1194, 1195, 1198, and 1200.

In yet another embodiment, the substituted pyrazolo[1,5-a]pyridine compound is a phosphodiesterase inhibitor.

In a particular embodiment, the compound possesses an IC₅₀ value based upon a PDE 10 inhibition assay as described herein of less than about 20.00 μM. In one embodiment, the compound possesses an IC₅₀ value based upon a PDE 10 inhibition assay ranging from about 1.0 to 20.0 μM, preferably from 1.0 to 10.0 μM. Illustrative compounds particularly effective in PDE-10 inhibition include 1137, 1134, 1136, 1153, 1154, 1158, 1164, 1165, 1166, 1173, 1196, 1198, 1199, and 1200.

In yet another embodiment, the compound possesses an IC₅₀ value based upon a PDE 4 inhibition assay as described herein of less than about 30.00 μM. In one embodiment, the compound possesses an IC₅₀ value based upon a PDE 10 inhibition assay ranging from about 1.0 to 20.0 μM, preferably from 1.0 to 10.0 μM. Illustrative compounds particularly effective in PDE-4 inhibition include 1137, 1134, 1136, 1153, 1154, 1155, 1156, 1158, 1164, 1168, 1173, 1174, 1175, 1176, 1177, 1178, 1182, 1183, 1194, 1195, 1196, 1198, and 1200.

In yet a further embodiment, a substituted pyrazolo[1,5-a]pyridine compound is capable of both phosphodiesterase and JNK inhibition—i.e., is capable of dual inhibition.

In a particular embodiment related to the foregoing, a substituted pyrazolo[1,5-a]pyridine compound is capable of inhibition of at least one of JNK 3 or JNK 2 and is capable of inhibition of at least one of PDE 10 or PDE 4. In a specific embodiment, the compound will possess (i) an IC₅₀ value in at least one of a JNK 3 or JNK 2 inhibition assay as described herein of less than about 5.00 μM, and (ii) an IC₅₀ value based upon at least one of a PDE 10 or PDE 4 inhibition assay as described herein of less than about 20.0 or less than about 30.0 μM, respectively. Particularly preferred compounds having the above features include 1137, 1134, 1136, 1153, 1154, 1156, 1158, 1164, 1165, 1166, 1167, 1168, 1173, 1174, 1175, 1176, 1177, 1178, 1180, 1182, 1183, 1184, 1194, 1195, 1198, and 1200. In yet an additional embodiment, a substituted pyrazolo[1,5-a]pyridine compound capable of dual inhibition (Le., phosphodiesterase and JNK) possesses an R₂ moiety that is hydrogen or lower cycloalkyl, an R₃ moiety that is pyrimidinyl-2-lowercycloalkylamine, and an R₇ moiety that is either hydrogen or methyl. In a related embodiment, in reference to structure III above, R₂ is cyclopropyl, R₁₀ is hydrogen and R₁₁ is isopropyl or cyclopropyl (i.e., a C3 linear or cyclic moiety) and R₇ is hydrogen. In yet another embodiment, R₂ is hydrogen, R₁₀ is hydrogen and R₁₁ is cyclopentyl and R₇ is methyl.

In yet another embodiment, in addition to the ability to inhibit both phosphodiesterases and JNK enzymes, a substituted pyrazolo[1,5-a]pyridine compound as provided herein is effective in treating neuropathic pain, as indicated by performance in a rat chronic constriction model as described herein. Examples of beneficial performance in a rat chronic construction model include values greater than 1.0 gram (see, e.g., Table 4).

In yet another embodiment, a substituted pyrazolo[1,5-a]pyridine compound as provided herein is capable of inhibiting glial cell activation. Particularly effective exemplary compounds capable of inhibiting glial cell activation, as indicated by results in a BV-2 mouse microglial cell assay as described herein include 1137, 1158, 1164, 1165, 1166, 1173, 1180, 1183, 1184, 1194, 1195, 1198, and 1200. In the assay examined, compounds were capable of inhibiting the cytokines TNF-α and/or MCP-1 in mouse BV-2 microglial cells activated with lipopolysaccharide (LPS) and IFN-γ (see, e.g., Table 3).

In a specific embodiment, the compound exhibits an EC₅₀ value for MCP-1 and/or TNF-α in a BV-2 glial cell assay as described herein of less than about 6.0 μM, e.g., from about 0.01 to about 6.0 μM. In a preferred embodiment, the compound exhibits an EC₅₀ value for MCP-1 and/or TNF-α in a BV-2 glial cell assay as described herein in a range from about 0.01 to 5.0 μM. In yet another embodiment, the compound exhibits an EC₅₀ value for MCP-1 and/or TNF-α in a BV-2 glial cell assay from about 0.01 to 1.5 μM.

The compounds provided herein are also effective at inhibiting JNK in additional cell types such as in human SH-SY5Y neuroblastoma cells and in E18 rat neuronal cells as demonstrated in in-vitro assays. Briefly, production of phosphorylated c-Jun was stimulated by addition of a stimulant such as 6-hydroxydopamine (6-OHDA) or amyloid beta peptide in the various cell types; test compound was added and the EC₅₀ values were determined on the ability to inhibit the production of phosphorylated c-JUN as measured by ELISA. In one embodiment, a substituted pyrazolo[1,5-a]pyridine compound as provided herein is capable of inhibiting phosphorylated c-Jun production in cells as indicated by an EC₅₀ value of less than about 10 μM as determined using a phosphorylated c-JUN assay as described herein. In yet another embodiment, a substituted pyrazolo[1,5-a]pyridine compound possesses an EC₅₀ value in a range of from about 0.05 to about 10 μM, and preferably in a range of from about 0.05 to about 8.0 μM. Representative values are shown in Table 3.

In yet another embodiment, a substituted pyrazolo[1,5-a]pyridine compound as described herein is water soluble.

In a further embodiment, a substituted pyrazolo[1,5-a]pyridine compound as provided herein possesses a half life of greater than one hour following oral dosing as measured in Sprague-Dawley rats. Even more preferably, a substituted pyrazolo[1,5-a]pyridine compound possesses a half life measured as described above of greater than 2 hours. Illustrative compounds having particularly long half lives include compounds 1173, 1180, 1195, 1198 and 1200. Compounds 1173 and 1198 and 1200 each have half lives greater than 3 hours, and are also capable of dual inhibition (i.e., multi-target activity against phosphodiesterase 4, 10 and JNK kinases 2 & 3). Particularly preferred are water soluble compounds capable of dual inhibition of both phosphodiesterases and c-JUN terminal kinases. An example of one such compound is 1200.

Also provided herein is a pharmaceutical composition comprising a substituted pyrazolo[1,5-a]pyridine compound and its pharmaceutically acceptable salts as described herein and a pharmaceutically acceptable carrier. Illustrative of the inventive formulation are those comprising a compound or its pharmaceutically acceptable salts selected from the following table and a pharmaceutically acceptable carrier.

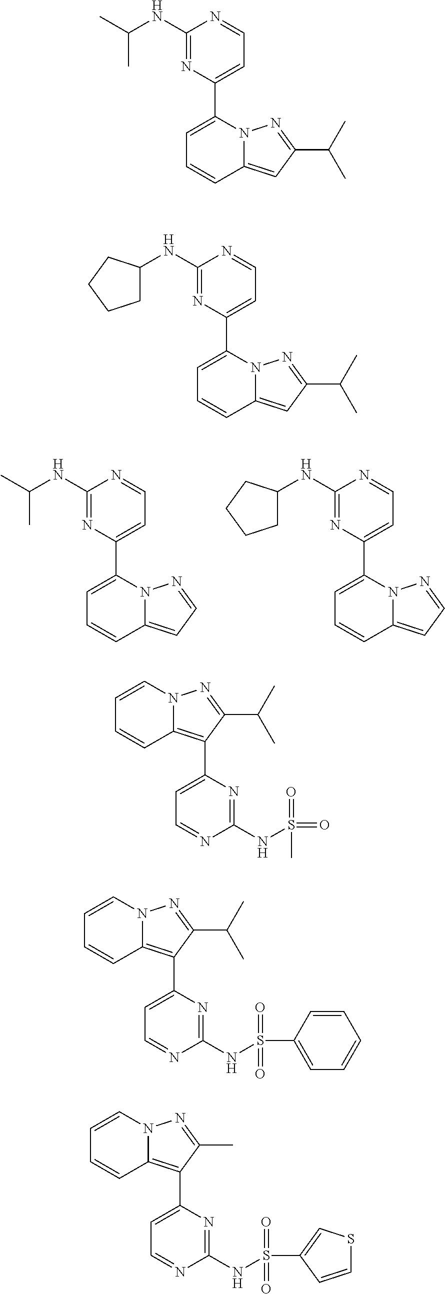

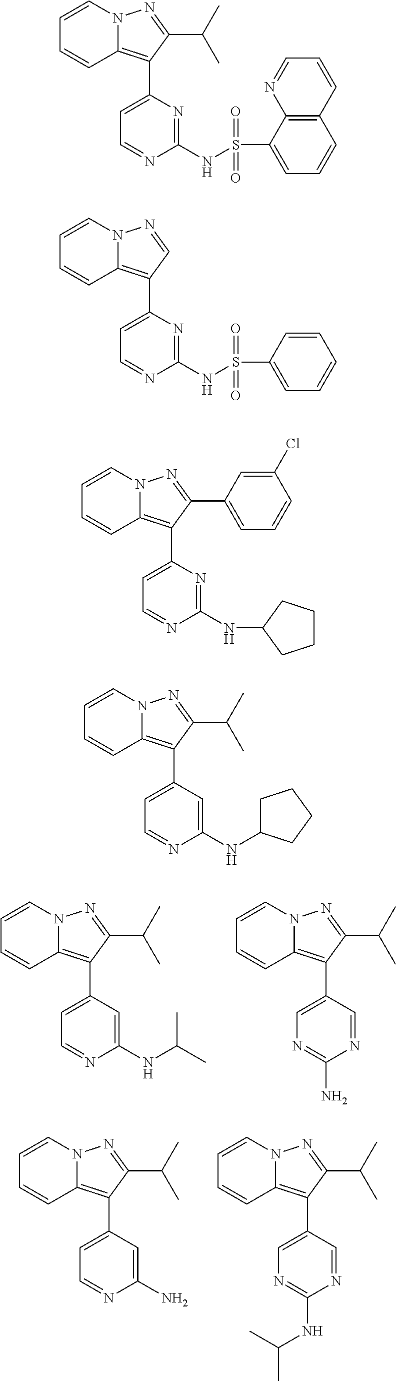

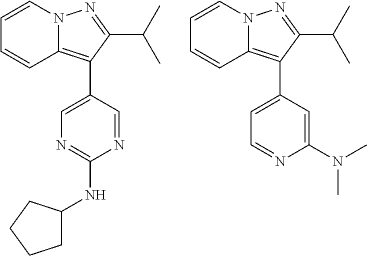

In yet another aspect, provided herein is a method for treating a neurodegenerative disease by administering one or more of the substituted pyrazolo[1,5-a]pyridine compounds described herein. Neurodegenerative diseases suitable for treatment with one or more of the substituted pyrazolo[1,5-a]pyridine compounds provided herein include Alzheimer's, Parkinson's, Huntington's, Lou Gehrig's, cerebral palsy, multiple sclerosis, narcolepsy, various dementias.

In yet another but related aspect, provided herein is use of any one or more of the instant substituted pyrazolo[1,5-a]pyridine compounds for treating a mammalian subject experiencing neuropathic pain.

In a particular embodiment, the subject is suffering from neuropathic pain associated with a condition selected from the group consisting of postherpetic neuralgia, trigeminal neuralgia, diabetic neuropathy, migraine, herpes, HIV, traumatic nerve injury, stroke, post-ischemia, fibromyalgia, reflex sympathetic dystrophy, complex regional pain syndrome, spinal cord injury, and cancer-chemotherapeutic-induced neuropathic pain.

In a further aspect, provides herein is a method for modulating glial cell activation by treatment with a substituted pyrazolo[1,5-a]pyridine compound as provided herein. Compounds particular effective in modulating glial cell activation possess an EC₅₀ value for MCP-1 and/or TNF-α in a BV-2 glial cell assay as described herein from about 0.01 to 1.5 μM. Preferred compounds for modulating glial cell activation include 1137, 1158, 1164, 1165, 1166, 1173, 1180, 1183, 1184, 1194, 1195, 1198, and 1200.

In yet another aspect, provided herein is a method for treating inflammation by administering to a subject suffering from an inflammatory condition a therapeutically effective amount of a substituted pyrazolo[1,5-a]pyridine compound as described herein. In a related embodiment, provided herein is use of a substituted pyrazolo[1,5-a]pyridine compound for treating inflammation. Inflammatory diseases or disorders suitable for treatment using one or more of the compounds provided herein include rheumatoid arthritis, osteoarthritis, systemic lupus erythematosus, Sjogren's syndrome, Crohn's disease, inflammatory bowel disease, pelvic inflammatory disease, and the like.

In yet another aspect, provided herein is a method of treating a tumor by administering a substituted pyrazolo[1,5-a]pyridine compound as described herein. Illustrative tumor types include gliomas, monocytic leukemias/lymphomas, and potentially certain other sarcomas and carcinomas.

Additional embodiments of the present method, compositions, and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

These and other objects and features of the invention will become more fully apparent when read in conjunction with the following detailed description.

### BRIEF DESCRIPTION OF THE DRAWINGS

FIGS. 1A.-1D show the chemical structures of various exemplary substituted pyrazolo[1,5-a]pyridine compounds having multi-target activity, i.e., phosphodiesterase and JNK kinase activity, as well as glial attenuation.

FIG. 2 demonstrates dose-dependent inhibition of amyloid beta-induced phosphorylation of JNK kinase by exemplary compound AV1184 as described in detail in Example 2.

FIG. 3 illustrates the results of treatment with an exemplary compound, AV1173, versus control in OHDA-lesioned rats in a model of Parkinson's disease as described in detail in Example 3.

FIG. 4 illustrates the cognitive enhancing properties of AV1137 in rats having a scopolamine-induced deficit in the Morris Maze Test in comparison to an amnesic control as described in Example 4.

FIG. 5 illustrates the reduced escape tendencies in normal rats (absent scopolamine treatment) treated with AV1137 relative to saline controls as described in Example 4.

FIG. 6 demonstrates the efficacy of AV1173 in the rat chronic constriction injury (CCI) model of neuropathic pain as described in Example 5.

### DETAILED DESCRIPTION

Various aspects of the invention now will be described more fully hereinafter. Such aspects may, however, be embodied in many different forms and should not be construed as limited to the embodiments set forth herein; rather, these embodiments are provided so that this disclosure will be thorough and complete, and will fully convey the scope of the invention to those skilled in the art.

The practice of the present disclosure will employ, unless otherwise indicated, conventional methods of chemistry, biochemistry, and pharmacology, within the skill of the art. Such techniques are explained fully in the literature. See, e.g.; A. L. Lehninger, Biochemistry (Worth Publishers, Inc., current addition); Morrison and Boyd, Organic Chemistry (Allyn and Bacon, Inc., current addition); J. March, Advanced Organic Chemistry (McGraw Hill, current addition); Remington: The Science and Practice of Pharmacy, A. Gennaro, Ed., 20^{th} Ed.; Goodman & Gilman The Pharmacological Basis of Therapeutics, J. Griffith Hardman, L. L. Limbird, A. Gilman, 10^{th} Ed.

All publications, patents and patent applications cited herein, whether supra or infra, are hereby incorporated by reference in their entirety.

### DEFINITIONS

It must be noted that, as used in this specification, the singular forms “a,” “an,” and “the” include plural referents unless the context clearly dictates otherwise. Thus, for example, reference to a “compound” includes a single compound as well as two or more of the same or compounds, reference to an “excipient” includes a single excipient as well as two or more of the same or different excipients, and the like.

In describing and claiming the present invention, the following terminology will be used in accordance with the definitions described below.

“Alkyl” refers to a hydrocarbon chain, typically ranging from about 1 to 20 atoms in length. Such hydrocarbon chains are preferably but not necessarily saturated and may be branched or straight chain, although typically straight chain is preferred. Exemplary alkyl groups include methyl, ethyl, propyl, isopropyl, butyl, pentyl, 1-methylbutyl, 1-ethylpropyl, 3-methylpentyl, and the like. As used herein, “alkyl” includes cycloalkyl when three or more carbon atoms are referenced.

“Lower” in reference to a particular functional group means a group having from 1-6 carbon atoms.

For example, “lower alkyl” refers to an alkyl group containing from 1 to 6 carbon atoms, and may be straight chain or branched, as exemplified by methyl, ethyl, propyl, isopropyl, 1-ethylpropyl, 1,2-dimethylpropyl, n-butyl, i-butyl, sec-butyl, t-butyl, and the like. “Cycloalkyl” refers to a saturated cyclic hydrocarbon chain, including bridged, fused, or spiro cyclic compounds, preferably made up of 3 to about 12 carbon atoms, more preferably 3 to about 8.

The term “alkylene” includes straight or branched alkylene chains such as methylene, ethylene, trimethylene, tetramethylene, pentamethylene, hexamethylene, and the like.

“Non-interfering substituents” are those groups that, when present in a molecule, are typically non-reactive with other functional groups contained within that molecule.

The term “substituted” as in, for example, “substituted alkyl” or “substituted aryl” refers to a moiety (e.g., an alkyl or aryl group) substituted with one or more non-interfering substituents, such as, but not limited to: C₃-C₈ cycloalkyl (e.g., cyclopropyl, cyclobutyl, and the like), halogen, (e.g., fluoro, chloro, bromo, and iodo), cyano, oxo, acyl, ester, sulfhydryl, amino, thioalkyl, carbonyl, carboxyl, carboxamido, alkoxy, lower alkyl, aryl, substituted aryl, phenyl, substituted phenyl, cyclic amides (e.g., cyclopentamide, cyclohexamide, etc., morpholinamide, tetrahydroquinolineamide, tetrahydroisoquinolineamide, coumarinamides, and the like). For substitutions on a phenyl ring, the substituents may be in any orientation (i.e., ortho, meta, or para).

“Alkoxy” refers to an —O—R group, wherein R is alkyl or substituted alkyl, preferably C₁-C₂₀ alkyl (e.g., methoxy, ethoxy, propoxy, isopropoxy, etc.), preferably C₁-C₇.

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl. Preferred aryl groups contain one or two aromatic rings.

“Heteroaryl” is an aryl group containing from one to four heteroatoms, preferably N, O, or S, or a combination thereof. Heteroaryl rings may also be fused with one or more cyclic hydrocarbon, heterocyclic, aryl, or heteroaryl rings. Exemplary heteroaryl rings include pyridine, pyridazine, pyrrole, pyrazole, triazole, imidazole, oxazole, isoxazole, thiazole, isothiazole, tetrahyquinoline, tetrahyquinolineamide, tetrahydroisoquinoline, tetrahydroisoquinolineamide, coumarin, courmarinamide, and the like.

“Heterocycle” or “heterocyclic” means one or more rings of 5-12 atoms, preferably 5-7 atoms, with or without unsaturation or aromatic character and having at least one ring atom which contains 1 to 4 heteroatoms independently selected from sulfur, oxygen, and nitrogen wherein the nitrogen and sulfur heteroatoms are optionally oxidized and the nitrogen heteroatom optionally quaternized, including bicyclic, and tricyclic ring systems.

“Amino” or “amine” as used herein, encompasses unsubstituted (—NH₂), mono-substituted amino and di-substituted amino compounds (relative to an unsubstituted amino group as a substituent on a core molecule such as a PYrazolo[1,5-a]pyridine). For example, amino refers to the moiety, —NRₐR_{b}, where Rₐ and R_{b} are each independently —H, —OH, —OC(O)NH₂, alkyl, cycloalkyl, aryl, or alkylaryl.

As used herein, the term “functional group” or any synonym thereof is meant to encompass protected forms thereof.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, non-toxic salts such as amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic carboxylic or sulfonic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Substantially” or “essentially” means nearly totally or completely, for instance, 95% or greater of some given quantity.

“Optional” or “optionally” means that the subsequently described circumstance may or may not occur, so that the description includes instances where the circumstance occurs and instances where it does not.

By “pathological pain” is meant any pain resulting from a pathology, such as from functional disturbances and/or pathological changes, lesions, burns, injuries, and the like. One form of pathological pain is “neuropathic pain” which is pain thought to initially result from nerve damage but extended or exacerbated by other mechanisms including glial cell activation. Examples of pathological pain include, but are not limited to, thermal or mechanical hyperalgesia, thermal or mechanical allodynia, diabetic pain, pain arising from irritable bowel or other internal organ disorders, endometriosis pain, phantom limb pain, complex regional pain syndromes, fibromyalgia, low back pain, cancer pain, pain arising from infection, inflammation or trauma to peripheral nerves or the central nervous system, multiple sclerosis pain, entrapment pain, and the like.

“Hyperalgesia” means an abnormally increased pain sense, such as pain that results from an excessive sensitiveness or sensitivity. Examples of hyperalgesia include but are not limited to cold or heat hyperalgesia.

“Hypalgesia” (or “hypoalgesia”) means the decreased pain sense.

“Allodynia” means pain sensations that result from normally non-noxious stimulus to the skin or body surface. Examples of allodynia include, but are not limited to, cold or heat allodynia, tactile or mechanical allodynia, and the like.

“Nociception” is defined herein as pain sense. “Nociceptor” herein refers to a structure that mediates nociception. The nociception may be the result of a physical stimulus, such as, mechanical, electrical, thermal, or a chemical stimulus. Nociceptors are present in virtually all tissues of the body.

“Analgesia” is defined herein as the relief of pain without the loss of consciousness. An “analgesic” is an agent or drug useful for relieving pain, again, without the loss of consciousness.

The term “central nervous system” or “CNS” includes all cells and tissue of the brain and spinal cord of a vertebrate. Thus, the term includes, but is not limited to, neuronal cells, glial cells, astrocytes, cerebrospinal fluid (CSF), interstitial spaces and the like.

“Glial cells” refer to various cells of the CNS also known as microglia, astrocytes, and oligodendrocytes.

The terms “subject”, “individual” or “patient” are used interchangeably herein and refer to a vertebrate, preferably a mammal. Mammals include, but are not limited to, murines, rodents, simians, humans, farm animals, sport animals and pets. Such subjects are typically suffering from or prone to a condition that can be prevented or treated by administration of a compound of the invention.

The term “about”, particularly in reference to a given quantity, is meant to encompass deviations of plus or minus five percent.

“Treatment” or “treating” of a particular condition includes: (1) preventing such a condition, i.e. causing the condition not to develop, or to occur with less intensity or to a lesser degree in a subject that may be exposed to or predisposed to the condition but does not yet experience or display the condition, (2) inhibiting the condition, i.e., arresting the development or reversing the condition.

The term “addiction” is defined herein as compulsively using a drug or performing a behavior repeatedly that increases extracellular dopamine concentrations in the nucleus accumbens. An addiction may be to a drug including, but not limited to, psychostimulants, narcotic analgesics, alcohols and addictive alkaloids such as nicotine, cannabinoids, or combinations thereof.

A subject suffering from an addiction experiences addiction-related behavior, cravings to use a substance in the case of a drug addiction or overwhelming urges to repeat a behavior in the case of a behavioral addiction, the inability to stop drug use or compulsive behavior in spite of undesired consequences (e.g., negative impacts on health, personal relationships, and finances, unemployment, or imprisonment), reward/incentive effects associated with dopamine release, and dependency, or any combination thereof.

Addiction-related behavior in reference to a drug addiction includes behavior resulting from compulsive use of a drug characterized by dependency on the substance. Symptomatic of the behavior is (i) overwhelming involvement with the use of the drug, (ii) the securing of its supply, and (iii) a high probability of relapse after withdrawal.

“Optional” or “optionally” means that the subsequently described circumstance may or may not occur, so that the description includes instances where the circumstance occurs and instances where it does not.

By “water soluble” is meant a compound that is soluble in water to an extent of at least 10 milligrams per milliliter in water at 25° C. and a pH 7.0.

Substituted Pyrazolo[1,5-a]pyridines

The present disclosure is directed to substituted pyrazolo[1,5-a]pyridines having a unique multi-target activity. Based upon both in-vitro and in-vivo assays, these compounds have been found to possess activity against both phosphodiesterases (PDE) and c-JUN kinases (JNK) (i.e., the compounds herein are “dual” inhibitors). More specifically, the subject compounds possess activity against PDE 4 and/or PDE 10 and JNK kinases 2 and/or 3. This unique dual activity makes such compounds effective in treating multiple indications including neurodegenerative/cognitive disorders, neuropathic pain, and in treating conditions involving modulation of glial cell activation, among others.

These and other features of the compounds will now be described in the sections which follow.

The substituted pyrazolo[1,5-a]pyridine compounds provided herein may generally be described as having the following structure. These compounds are referred to generally as pyrazolo[1,5-a]pyridine compounds, where the numbering of the non-bridgehead ring atoms is shown in structure I.

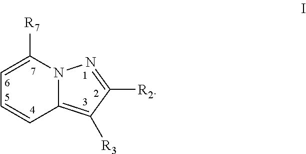

The compounds typically possess a substituent at the 3-ring position, and may also possess a substituent at the 2-ring and/or 7-ring position. A compound may possess a single substituent at R₃ (i.e., is mono-substituted), or may possess substituents at positions R₂ and R₃, or may possess substituents at positions R₃ and R₇ (i.e., is di-substituted), or may possess substituents at each of R₂, R₃, and R₇ (i.e., is tri-substitued). That is to say, compounds provided herein include 2,3 substituted pyrazolo[1,5-a]pyridines, 3-substituted pyrazolo[1,5-a]pyridines, 3,7-substituted pyrazolo[1,5-a]pyridines, and 2,3,7-substituted pyrazolo[1,5-a]pyridines. Generally, in reference to structure I, R₃ is an amino-substituted pyrimidine or pyridine; R₂ is independently H or an organic radical selected from the group consisting of alkyl, cycloalkyl, alkoxyalkyl (e.g., compound 1117, methoxymethyl), aryl (e.g., phenyl), and haloaryl; and R₇ is independently selected from H or alkyl. The presence of a cycloalkyl, e.g., a cyclopropyl, group at this position for certain exemplary compounds results in an unexpected increase in oral bioavailability and increased blood levels, as shown in the supporting examples.

When R₃ is an amino-substituted pyrimidine, the amine substituent is positioned at the 2-ring position of the pyrimidine (i.e., at the carbon interposed between the two ring nitrogens of the pyrimidine ring). That is to say, the amino group is located at the 2 position of the pyrimidine ring, and the pyrimidine is attached at its 4-position to the pyrazolo[1,5-a]pyridine core. More particularly, when the 3-substituent of the pyrazolo[1,5-a]pyridine is a substituted pyrimidin-2-amine moiety, the pyrimidine is attached to the core system via the 4-position of the pyrimidine. (See exemplary structure below, where R₁₀ and R₁₁ are each independently selected from H, alkyl, substituted alkyl, cycloalkyl, and aliphatic 3, 4, 5, and 6-membered nitrogen containing heterocycles).

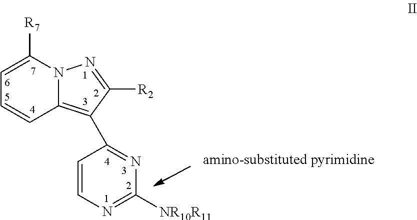

Alternatively when R₃ is an amino-substituted pyridine, R₃ is a pyridine ring possessing an amine substituent at its 2-ring position as illustrated in structure III, while the pyridine ring is connected at its 5-ring position to the pyrazolo[1,5-a]pyridine core.

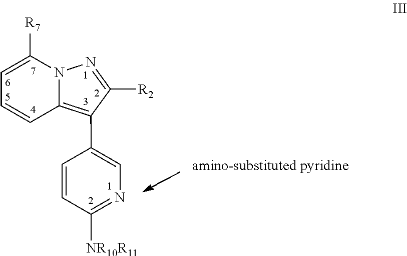

In one embodiment, R₃ is an amino-substituted pyridine as illustrated in structure IV.

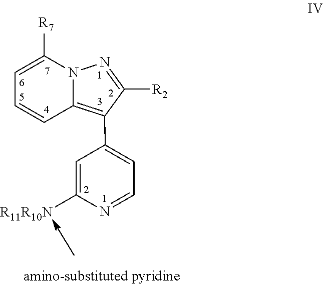

For structures II, III, and IV, illustrative amine substituents possess the structure, —NR₁₀R₁₁, where R₁₀ and R₁₁ are each independently selected from H, alkyl, substituted alkyl, S(O)₂R′, cycloalkyl, and aliphatic 3, 4, 5, and 6-membered nitrogen containing heterocycles. When R₁₀ or R₁₁ is S(O)₂R′, R′ is selected from the group consisting alkyl, aryl and heteroaryl. For example, R′ can be methyl, ethyl, propyl, phenyl, thiophene or quinoline.

In an aspect of the invention, the amine substituent on either the pyrimidine ring or the pyridine ring is a mono-substituted amine where one of R₁₀ or R₁₁ is hydrogen. For instance, the amine substituent in either structure II, III, or IV is one where R₁₀ is hydrogen and R₁₁ is lower alkyl, substituted lower alkyl, or lower cycloalkyl. Examples of R₁₁ substituents include methyl, ethyl, propyl, isopropyl, butyl, 2-methylpropyl, 3-hydroxypropyl, pentyl, N-3-pentyl, 1-methylbutyl, 1-ethylpropyl, 3-methylpentyl, cyclopropyl, cyclobutyl, cyclopentyl, and the like. See, e.g., compounds 1137, 1134, 1136, 1153, 1154, 1155, 1156, 1157, 1158, 1159, 1164, 1165, 1166, 1167, 1168, 1173, 1174, 1175, 1176, 1177, 1178, 1179, 1180, 1182, 1183, 1184, 1194, 1195, 1197, 1198, 1199 and 1200.

Alternatively, the amine substituent on either the pyrimidine ring or the pyridine ring is an unsubstituted amine where both R₁₀ or R₁₁ are hydrogen. See, e.g., compounds 1139 and 1196.

Still in reference to R₃, the amine substituent in either structure II, III, or IV may possess R₁₀ as hydrogen, where R₁₁ is an aliphatic 3, 4, 5, and 6-membered nitrogen containing heterocycle selected from aziridine, pyrrolidine, and piperidine. One example is a compound according to structure II or III where R₁₁ is a pyrrolidine ring connected to the amine nitrogen at the 3-ring position of the pyrrolidine. See, e.g., compound 1159.

Illustrative amino substituents corresponding to either structure II, III or IV include the following, where the squiggly line indicates attachment to the corresponding pyrimidine or pyridine:

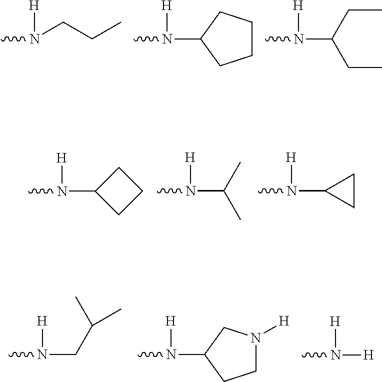

Representative R₃ substituents include:

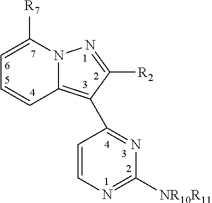

where the amine substituent, —NR₁₀R₁₁ is selected from:

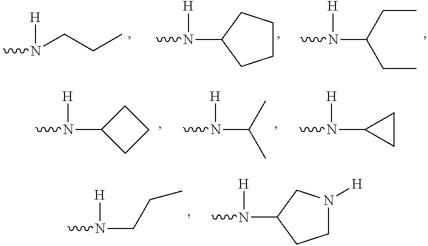

and R₂ and R₇ are as described generally above. See Table 1.

Additional R₃ substituents are shown in the following structures, where R₂ and R₇ are as described elsewhere herein:

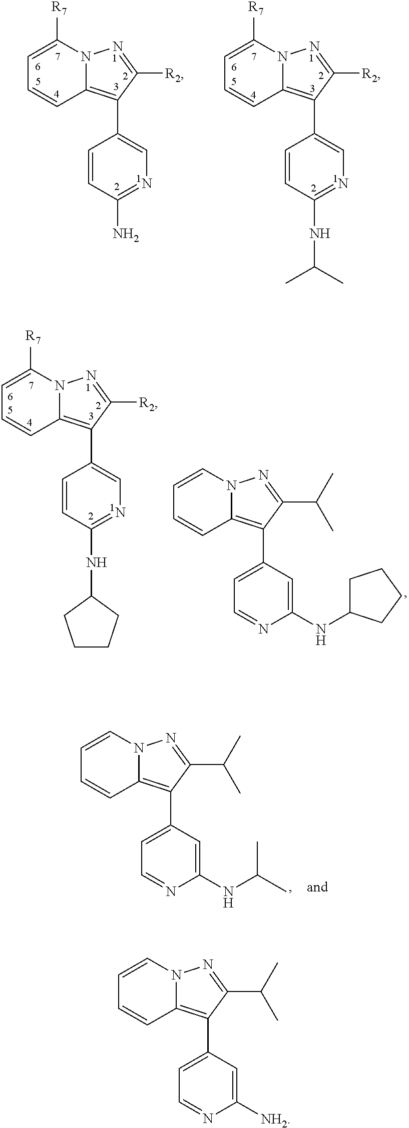

In reference to the subject compounds, turning now to substituent R₇, typically, R₇ is either hydrogen or lower alkyl, e.g., is selected from methyl, ethyl, propyl, isopropyl, 1-ethylpropyl, 1,2-dimethylpropyl, n-butyl, i-butyl, sec-butyl, t-butyl, and the like. Typically, when R₇ is lower alkyl, R₇ is methyl. See, e.g., 1168, 1183 and 1184.

Turning now to R₂, as described above, R₂ typically is independently H or an organic radical selected from the group consisting of alkyl, cycloalkyl, alkoxyalkyl (e.g., compound 1117, methoxymethyl), aryl (e.g., phenyl), and haloaryl. In several of the representative compounds, R₂ is lower alkyl or lower cycloalkyl. Illustrative lower alkyl R₂ groups include methyl, ethyl, propyl, isopropyl, 1-ethylpropyl, 1,2-dimethylpropyl, n-butyl, i-butyl, sec-butyl, t-butyl, and the like. Lower cycloalkyl groups are selected from cyclopropyl, cyclobutyl, and cyclopentyl. See, e.g., compounds 1137, 1134, 1135, 1136, 1139, 1153, 1154, 1155, 1156, 1157, 1158, 1159, 1168, 1173, 1174, 1177, 1178, 1182, 1196, 1197, 1198, 1199, and 1200. Two preferred R₂ groups include isopropyl and cyclopropyl.

Alternatively, R₂ can be phenyl or halo-substituted phenyl. A halo-substituted phenyl generally corresponds to a phenyl ring having a single halogen substituent selected from fluorine, chlorine or bromine or iodine, preferably chlorine or fluorine. The halogen may be at any position on the phenyl ring, e.g., alpha, meta, or para to the parent pyrazolo[1,5-a]pyridine core structure. In one particular embodiment, the halogen is at the 3-position of the phenyl ring (assuming that the 1-position of the phenyl is the attachment to the core). Compounds such as these include 1176, 1179, 1180, 1194 and 1195.

In yet another embodiment, R₂ is an alkyl alkoxy group, preferably a lower alkyl lower alkoxy group. Illustrative R₂ substituents falling into this classification include methyl methoxy (—CH₂OCH₃), ethyl methoxy (—CH₂CH₂OCH₃), and the like. For instance, a lower alkyl lower alkoxy substituent may be described as —R₁₂—O—R₁₃, where R₁₂ and R₁₃ are each selected from lower alkyl, and R₁₂ is attached to the parent pyrazolo[1,5-a]pyridine core structure. An R₁₂ group may be a linear lower alkyl such as methyl, ethyl, propyl, butyl, pentyl, or hexyl, while R₁₃ taken together with the adjacent oxygen may be linear or branched alkoxy. Illustrative R₁₃ groups include methyl, ethyl, propyl, isopropyl, 1-ethylpropyl, 1,2-dimethylpropyl, n-butyl, butyl, sec-butyl, t-butyl, and the like. See, e.g., compound 1175.

Exemplified R₂ groups include hydrogen, methyl, isopropyl, tert-butyl, cyclopropyl, butyl, methyl methoxy, phenyl, sec-butyl, 3-fluorophenyl, and 3-chorophenyl.

Compounds provided herein are intended to encompass the parent pyrazolo[1,5-a]pyridine core structure substituted with any combination of R₂, R₃, and R₇ moieties as described herein, as consistent with the general features provided.

In certain instances, in reference to structure I, if the compound is a 2,3-substituted pyrazolo[1,5-a]pyridine and R₂ is isopropyl, then when R₃ is a substituted or unsubstituted (referring to the amine moiety) pyrimidin-2-amine moiety substituted at the 4-position of the pyrimidine ring to the pyrazolo[1,5-a]pyridine, R₃ is other than isopropylpyrimidin-2-amine (1137), pyrimidin-2-amine (1139), (pyrimidin-2-ylamino)propan-1-ol (1134) and 3-(piperazin-1-yl)propyl)pyrimidin-2-amine (1135). For example, in such particular embodiment, R₃ is other than

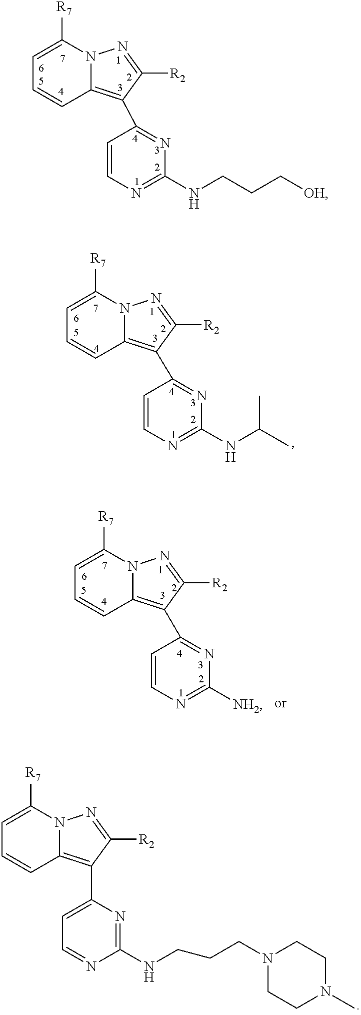

Illustrative compounds having values for R₂, R₃ and R₇ as described above are provided in Table I and in the accompanying examples.

As stated previously, a reference to any one or more of the herein-described substituted pyrazolo[1,5-a]pyridines is meant to encompass, where applicable, any and all enantiomers, mixtures of enantiomers including racemic mixtures, prodrugs, pharmaceutically acceptable salt forms, hydrates (e.g., monohydrates, dihydrates, etc.), solvates, different physical forms (e.g., crystalline solids, amorphous solids), and metabolites.

The substituted pyrazolo[1,5-a]pyridine compounds provided are prepared using conventional synthetic organic chemistry techniques known to those skilled in the art of organic synthetic chemistry and methodology.

### Features

As described above, in addition to possessing a myriad of features, it has been discovered that the compounds provided herein possess a unique multi-target activity not observed in known selective Jun N-terminal kinase inhibitors such as SP600125 (Bennet, B., et al, PNAS, Nov. 20, 2001, 98 (24), p. 13681), nor in the opioid alkaloid, papavarine, a selective phosphodiesterase inhibitor for the PDE10A subtype found mainly in the striatum of the brain (Boswell-Smith, V., et al., Br J. Pharmacol. 2006 January; 147(S1): S252-S257), nor in the selective PDE IV inhibitor, rolipram (Liang, L., et al., Diabetes, Vol 47, Issue 4 570-575). See Table 2. Thus, in contrast to the foregoing known compounds, it has been discovered that the instant compounds are dual inhibitors, i.e., they inhibit both phosphodiesterases (such as PDE 10 and PDE 4) and Jun N-terminal kinases (e.g., JNK 3 and JNK 2). These targets are quite different—a PDE inhibitor functions to block one or more of the subtypes of the enzyme phosphodiesterase, thereby preventing the inactivation of cAMP and cGMP by the PDE subtype, while a JNK inhibitor prevents binding and phosphorylation of c-Jun on Ser63 and Ser73 within its transcriptional domain, thereby impacting response to stress stimuli, T-cell differentiation, apoptosis, and the like. This feature of the compounds, i.e., their multi-target PDE and JNK activity, makes such compounds useful for treating multiple and varied indications including neurodegenerative diseases, inflammatory disorders, certain tumors, in addition to neuropathic pain, opiate withdrawal and addiction, modulation of glial cell activation, etc.

### JNK Inhibition.

As stated above, the instant compounds are JNK-inhibitors, i.e., are capable of inhibiting either JNK-2 or JNK-3 enzyme. See Table 2. Typically, the substituted pyrazolo[1,5-a]pyridine compounds will possess an IC₅₀ value based upon a JNK inhibition assay as described herein of less than about 5.00 μM.

With respect to JNK-3 inhibition, a compound will preferably possess an IC₅₀ value based upon a JNK 3 inhibition assay as described herein ranging from about 0.01 to 5.00 μM, preferably from 0.01 to 4.00 μM or more preferably from about 0.01 to about 3.00 μM. Particularly preferred are compounds having an IC₅₀ value based upon a JNK 3 inhibition assay in a range from 0.01 to 2.00 μM. Illustrative compounds particularly effective in JNK-3 inhibition include 1136, 1158, 1164, 1165, 1166, 1167, 1173, 1174, 1175, 1176, 1177, 1179, 1180, 1182, 1183, 1184, 1194, 1195, 1198, and 1200.

With respect to JNK-2 inhibition, a compound will typically possess an IC₅₀ value based upon a JNK 2 inhibition assay as described herein ranging from about 0.01 to 5.00 μM, preferably from 0.01 to 3.00 μM or more preferably from about 0.01 to about 2.00 μM. Particularly preferred are compounds having an IC₅₀ value based upon a JNK 2 inhibition assay in a range from 0.01 to 2.00 μM. Illustrative compounds particularly effective in JNK-2 and falling within this classification inhibition include 1153, 1156, 1164, 1165, 1166, 1167, 1173, 1174, 1176, 1194, 1195, 1198, and 1200.

With respect to inhibition of both JNK 2 and JNK 3, several of the compounds tested were discovered to be inhibitors of both JNK 2 and JNK 3. In certain instances, a substituted pyrazolo[1,5-a]pyridine compound will possess IC₅₀ values in both JNK 2 and JNK 3 inhibition assays as described herein ranging from about 0.01 to 2.00 vtM. Exemplary compounds exhibiting the foregoing feature include 1137, 1164, 1165, 1166, 1167, 1173, 1174, 1176, 1177, 1194, 1195, 1198, and 1200.

Certain of the compounds provided herein are also effective at inhibiting JNK in additional cell types such as in human SH-SY5Y neuroblastoma cells and in E18 rat neuronal cells as demonstrated in in-vitro assays. In the assays carried out, production of phosphorylated c-Jun was stimulated by addition of a stimulant such as 6-hydroxydopamine (6-OHDA) or amyloid beta peptide in the various cell types; test compound was added and the EC₅₀ values were determined. See, e.g., Example 2. For example, in one embodiment, a preferred substituted pyrazolo[1,5-a]pyridine compound as provided herein is capable of inhibiting phosphorylated c-Jun production in cells as indicated by an EC₅₀ value of less than about 10 vtM as determined using a phosphorylated c-JUN assay as described herein. Even more preferably, a substituted pyrazolo[1,5-a]pyridine compound possesses an EC₅₀ value in a range of from about 0.05 to about 10 μM, and most preferably in a range of from about 0.05 to about 8.0 μM.

### PDE Inhibition.

The instant substituted pyrazolo[1,5-a]pyridine compounds also act as inhibitors of phosphodiesterase, e.g., PDE 10 and PDE 4. In reference to Table 2 with respect to PDE 10, generally, a compound possesses an IC₅₀ value based upon a PDE 10 inhibition assay as described herein of less than about 20.00 μM. In one embodiment, the compound possesses an IC₅₀ value based upon a PDE 10 inhibition assay ranging from about 1.0 to 20.0 μM, preferably from 1.0 to 10.0 μM. Illustrative compounds particularly effective in PDE-10 inhibition include 1137, 1134, 1136, 1153, 1154, 1158, 1164, 1165, 1166, 1173, 1196, 1198, 1199, and 1200.

With respect to PDE 4, a compound will generally possess an IC₅₀ value based upon a PDE 4 inhibition assay as described herein of less than about 30.00 OA. Preferably, the compound possesses an IC₅₀ value based upon a PDE 10 inhibition assay ranging from about 1.0 to 20.0 μM, and even more preferably from 1.0 to 10.0 μM. Illustrative compounds particularly effective as PDE-4 inhibitors include 1137, 1134, 1136, 1153, 1154, 1155, 1156, 1158, 1164, 1168, 1173, 1174, 1175, 1176, 1177, 1178, 1182, 1183, 1194, 1195, 1196, 1198, and 1200.

In yet a further embodiment, a substituted pyrazolo[1,5-a]pyridine compound is capable of both phosphodiesterase and JNK inhibition—i.e., is capable of dual inhibition.

A feature of a preferred substituted pyrazolo[1,5-a]pyridine compound is its ability to act as an inhibitor of at least one of JNK 3 or JNK 2, as well as inhibit at least one of PDE 10 or PDE 4. For such compounds, the compound will typically possess (i) an IC₅₀ value in at least one of a JNK 3 or JNK 2 inhibition assay as described herein of less than about 5.00 μM, and (ii) an IC₅₀ value based upon at least one of a PDE 10 or PDE 4 inhibition assay as described herein of less than about 20.0 or less than about 30.0 μM, respectively. Particularly preferred and effective dual inhibitors include 1137, 1134, 1136, 1153, 1154, 1156, 1158, 1164, 1165, 1166, 1167, 1168, 1173, 1174, 1175, 1176, 1177, 1178, 1180, 1182, 1183, 1184, 1194, 1195, 1198, and 1200. In considering the foregoing compounds, it can be seen that in a particular embodiment, a substituted pyrazolo[1,5-a]pyridine compound capable of dual inhibition (i.e., phosphodiesterase and JNK) possesses an R₂ moiety that is hydrogen or lower cycloalkyl, an R₃ moiety that is pyrimidinyl-2-lowercycloalkylamine, and an R₇ moiety that is either hydrogen or methyl. In a particular instance, a substituted pyrazolo[1,5-a]pyridine compound capable of dual inhibition possesses, in reference to structure III above, R₂ that is cyclopropyl, R₁₀ that is hydrogen, R₁₁ that is isopropyl or cyclopropyl (i.e., a C3 linear or cyclic moiety) and R₇ that is hydrogen. In yet a father example of a dual inhibitor, R₂ is hydrogen, R₁₀ is hydrogen and R₁₁ is cyclopentyl and R₇ is methyl.

### Glial Cell Regulation.

Moreover, certain of the instant compounds are capable of inhibiting glial cell activation. Particularly effective exemplary compounds capable of inhibiting glial cell activation, as indicated by results in a BV-2 glial cell assay as described herein include 1137, 1158, 1164, 1165, 1166, 1173, 1180, 1183, 1184, 1194, 1195, 1198, and 1200. In the assay examined, compounds were capable of inhibiting the cytokines TNF-α and/or MCP-1 in mouse BV-2 microglial cells activated with lipopolysaccharide (LPS) and IFN-γ (see, e.g., Table 3). Thus, such compounds are particularly effective in inhibiting stimulant-induced cytokine production, thus providing an indication of their efficacy in treating inflammatory conditions.

In considering generally the data in Table 3, preferred compounds capable of glial cell modulation exhibit an EC₅₀ value for MCP-1 and/or TNF-α in a BV-2 glial cell assay as described herein of less than about 6.0 μM, e.g., from about 0.01 to about 6.0 μM. In a preferred embodiment, a compound exhibits an EC₅₀ value for MCP-1 and/or TNF-α in a BV-2 glial cell assay as described herein in a range from about 0.01 to 5.0 μM. Even more preferably, for a compound capable of glial cell modulation, the compound exhibits an EC₅₀ value for MCP-1 and/or TNF-α in a BV-2 glial cell assay from about 0.01 to 1.5 μM.

### Neuropathic Pain.

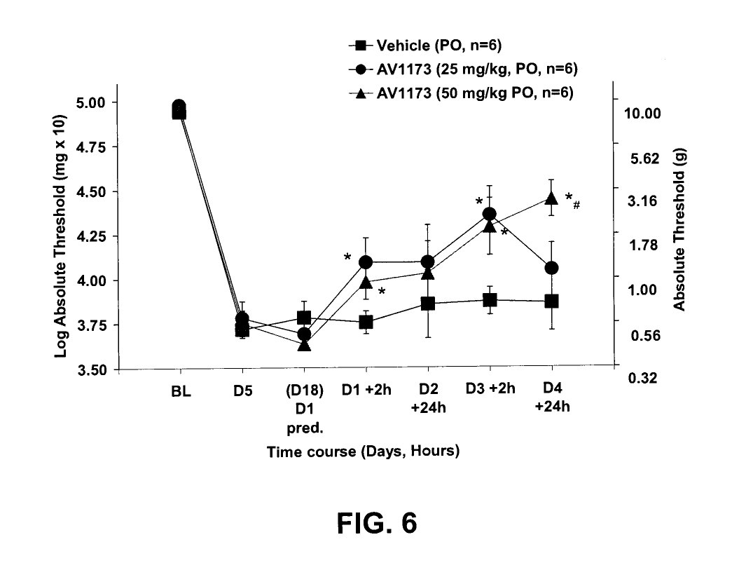

The instant compounds are surprisingly effective in providing a measurable reduction in the severity of neuropathic pain, and in particular, in providing a measurable reduction in the severity of certain manifestations of neuropathic pain such as mechanical allodynia. See, e.g., Example 5, where representative compounds were capable of reversing allodynia and sustaining efficacy overnight. Also see Table 3 and FIG. 6. Such relief of this neuropathy is achieved at well tolerated doses wherein general anesthesia is not observed and is, hence, specific and clinically relevant.

### Pharmacokinetics.

Ideally, a preferred substituted pyrazolo[1,5-a]pyridine compound as provided herein possesses a half life of greater than one hour following oral dosing as measured in a suitable in-vivo model such as in Sprague-Dawley rats. Even more preferably, a substituted pyrazolo[1,5-a]pyridine compound possesses a half life measured as described above of greater than 2 hours. Illustrative compounds having particularly prolonged half lives include compounds, 1173, 1180, 1195, 1198 and 1200. Compounds 1173 and 1198 and 1200 each have half lives greater than 3 hours, and are also capable of dual inhibition (i.e., multi-target activity against phosphodiesterase 4, 10 and JNK kinases 2 & 3). Particularly preferred are water soluble compounds capable of dual inhibition of both phosphodiesterases and c-JUN terminal kinases. An example of one such compound is 1200.

### Uses

Based upon the foregoing, the substituted pyrazolo[1,5-a]pyridines may be useful for treating a number of varied indications, diseases and disorders. Based upon the pharmacological and other data provided herein, it is believed that the compounds of the invention are particularly effective in treating one or more of the following conditions.

Based upon neuropathic pain indicator data, it can be seen that the compounds are useful in treating neuropathic pain. For example, the subject compounds may be used to treat neuropathic pain associated with certain syndromes such as viral neuralgias (e.g., herpes, AIDS), diabetic neuropathy, phantom limb pain, stump/neuroma pain, post-ischemic pain (stroke), fibromyalgia, reflex sympathetic dystrophy (RSD), complex regional pain syndrome (CRPS), cancer pain, vertebral disk rupture, spinal cord injury, and trigeminal neuralgia, cancer-chemotherapy-induced neuropathic pain, and migraine, among others. Given the potential for broader anti-inflammatory activity, other inflammatory conditions such as rheumatoid arthritis, osteoarthritis, autoimmune illnesses and even sepsis are likely indicated for clinical intervention with such compounds.

Additionally, based upon their ability to function as glial cell modulators, the subject compounds may be used for treating opiate tolerance and withdrawal, and/or as antiviral agents. The compounds may also be used in treating depression. Opioid-driven progressive glial activation causes glia to release neuroexcitatory substances, including the proinflammatory cytokines interleukin-1 (IL-1), tumor necrosis factor (TNF), and interleukin-6 (IL-6). These neuroexcitatory substances counteract the pain-relieving actions of opioids, such as morphine, and drive withdrawal symptomology, as demonstrated by experiments involving co-administration or pro- or anti-inflammatory substances along with morphine. Indeed, if morphine analgesia is established and then allowed to dissipate, potent analgesia can be rapidly reinstated by injecting IL-1 receptor antagonist, suggesting that dissipation of analgesia is caused by the activities of pain-enhancing proinflammatory cytokines rather than dissipation of morphine's analgesic effects.

The activity of other opioids may also be opposed by activation of glia. Studies show that glia and proinflammatory cytokines compromise the analgesic effects of methadone, at least in part, via non-classical opioid receptors ((i) Hutchinson, M. et al., and K. Johnson. Reduction of opioid withdrawal and potentiation of acute opioid analgesia by AV411 (ibudilast). Brain Behay. Immunity January 09; (ii) Hutchinson' M, Bland S, Johnson K, Rice K, Maier S, and Watkins L. Opioid-induced glial activation: Mechanisms of activation and implications for opioid analgesia, dependence and reward. NIDA-requested review in The Scientific World Journal 7:2007; (iii) Hutchinson, M., Johnson, K., and Watkins, L. Glial Dysregulation of Pain and Opioid Actions. in “Pain 2008 —An updated review”, J. M. Castro-Lopes, S. Raja, and M. Schmelz (eds). IASP Press, Seattle, 2008.

These results suggest that glia and proinflammatory cytokines will be involved in methadone withdrawal, and likely withdrawal from other opioids as well. These data also expand the clinical implications of glial activation, since cross-tolerance between opioids may be explained by the activation of the glial pain facilitatory system, which undermines all attempts to treat chronic pain with opioids. Since opioids excite glia, which in turn release neuroexcitatory substances (such as proinflammatory cytokines) that oppose the effects of opioids and create withdrawal symptoms upon cessation of opioid treatment, then compounds that suppress such glial activation, such as those provided herein, may also then be beneficial novel therapeutics for treatment of opioid withdrawal.\

Further, the compounds can be used for suppressing the release of dopamine in the nucleus accumbens of a subject. Dopamine release in the nucleus accumbens is thought to mediate the “reward” motivating drug use and compulsive behavior associated with addictions. Thus, the instant compounds may be used to attenuate or abolish the dopamine mediated “reward” associated with addictions, thus diminishing or eliminating cravings associated with addictions and the accompanying addiction-related behavior and withdrawal syndromes of a subject. (Bland, S T, et al., and Johnson, K. The glial activation inhibitor AV411 reduces morphine-induced nucleus accumbens dopamine release. BBI, March 2009).

For example, a therapeutically effective amount of a pyrazolo[1,5-a]pyridine compound may be administered to a subject to treat a drug addiction. The subject may be addicted to one or more drugs including, but not limited to, psychostimulants, narcotic analgesics, alcohols and addictive alkaloids, such as nicotine, cannabinoids, or combinations thereof. Exemplary psychostimulants include, but are not limited to, amphetamine, dextroamphetamine, methamphetamine, phenmetrazine, diethylpropion, methylphenidate, cocaine, phencyclidine, methylenedioxymethamphetamine and pharmaceutically acceptable salts thereof. Exemplary narcotic analgesics include, but are not limited to, alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, metazocine, methadone, metopon, morphine, opium extracts, opium fluid extracts, powdered opium, granulated opium, raw opium, tincture of opium, oxycodone, oxymorphone, pethidine, phenazocine, piminodine, racemethorphan, racemorphan, thebaine and pharmaceutically acceptable salts thereof. Addictive drugs also include central nervous system depressants, including, but not limited to, barbiturates, chlordiazepoxide, and alcohols, such as ethanol, methanol, and isopropyl alcohol.

The compounds may also be used to treat a behavior addition by administering a therapeutically effective amount of one or more of the subject compounds. A behavioral addiction can include, but is not limited to, compulsive eating, drinking, smoking, shopping, gambling, sex, and computer use. Addiction-related behavior in reference to a drug addiction includes behavior resulting from compulsive use of a drug characterized by dependency on the substance. Symptomatic of the behavior is (i) overwhelming involvement with the use of the drug, (ii) the securing of its supply, and (iii) a high probability of relapse after withdrawal. Thus, the compounds provided herein may be useful for treating addiction-related behavior as described above.

Certain compounds are also effective inhibitors of cytokine production. Based upon their ability to inhibit the production of stimulant-induced production of TNF-α and MCP-1, the compounds may also be used for treating any of a number of inflammatory conditions. Representative inflammatory disorders that may be treated by administering a compound as described herein include rheumatoid arthritis, bronchitis, tuberculosis, chronic cholecystitis, inflammatory bowel disease, acute pancreatitis, sepsis, asthma, chronic obstructive pulmonary disease, dermal inflammatory disorders such as psoriasis and atopic dermatitis, systemic inflammatory response syndrome (SIRS), acute respiratory distress syndrome (ARDS), cancer-associated inflammation, reduction of tumor-associated angiogenesis, osteoarthritis, diabetes, treatment of graft v. host disease and associated tissue rejection, Crohn's disease, delayed-type hypersensitivity, immune-mediated and inflammatory elements of CNS disease; e.g., Alzheimer's, Parkinson's, multiple sclerosis, etc. See, e.g., Example 3, which describes the efficacy of an exemplary substituted pyrazolo[1,5-a]pyridines compound in a standard rat model of Parkinson's.

As described in detail above, the instant compounds function as phosphodiesterase inhibitors. Phosphodiesterases regulate the intracellular levels of the secondary messengers, cAMP and cGMP, which affects cellular signaling. Therapeutic indications for PDE inhibitors such as those provided herein include hypertension, congestive heart failure, thrombosis, glaucoma, asthma, autoimmune disease and inflammation. Thus, any one or more of the foregoing conditions may be treated by administering a pyrazolo[1,5-a]pyridines compound provided herein.

Based upon their ability to prevent activation of JNKs, the compounds provided herein are useful as neuroprotective agents. The neuroprotective features of illustrative compounds are supported by Examples 3 and 4. Example 3 describes the utility of a representative compound in a standard rat model of Parkinson's disease, where administration of the compound was effective in reducing the rotational behavior of rats relative to those dosed with a vehicle control. Further, the utility of the subject compounds in treating cognitive disorders is exemplified in Example 4. Example 4 provides the results of a Morris water maze test, in which the cognitive enhancing effects of a representative compound are described.

The compounds provided herein may also be used to treat or prevent acute or subchronic pain by administration of an effective amount of a phosphodiesterase inhibitor or glial attenuator, such as the illustrative compounds provided herein, in combination with an opioid analgesic. The substituted pyrazolo[1,5-a]pyridine compound administered is effective to potentiate opioid-induced analgesia in the subject.

### Administration.

The compounds may be administered either systemically or locally. Such routes of administration include but are not limited to, oral, intra-arterial, intrathecal, intraspinal, intramuscular, intraperitoneal, intravenous, intranasal, subcutaneous, and inhalation routes.

More particularly, the compounds provided herein may be administered for therapeutic use by any suitable route, including without limitation, oral, rectal, nasal, topical (including transdermal, aerosol, buccal and sublingual), vaginal, parenteral (including subcutaneous, intramuscular, intravenous and intradermal), intrathecal, and pulmonary. The preferred route will, of course, vary with the condition and age of the recipient, the particular condition being treated, and the specific combination of drugs employed, if any.

One preferred mode of administration (depending upon the particular condition being treated) is directly to neural tissue such as peripheral nerves, the retina, dorsal root ganglia, neuromuscular junction, as well as the CNS, e.g., to target spinal cord glial cells by injection into, e.g., the ventricular region, as well as to the striatum (e.g., the caudate nucleus or putamen of the striatum), spinal cord and neuromuscular junction, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). A particularly preferred method for targeting spinal cord glia is by intrathecal delivery, rather than into the cord tissue itself.

Another preferred method for administering a substituted pyrazolo[1,5-a]pyridine-based composition is by delivery to dorsal root ganglia (DRG) neurons, e.g., by injection into the epidural space with subsequent diffusion to DRG. For example, such compositions can be delivered via intrathecal cannulation under conditions effective to diffuse the composition to the DRG. See, e.g., Chiang et al., Acta Anaesthesiol. Sin. (2000) 38:31-36; Jain, K. K., Expert Opin. Investig. Drugs (2000) 9:2403-2410.

Yet another mode of administration to the CNS uses a convection-enhanced delivery (CED) system. In this way, the compounds of the invention can be delivered to many cells over large areas of the CNS. Any convection-enhanced delivery device may be appropriate for delivery of a substituted pyrazolo[1,5-a]pyridine.

### Dose

Therapeutic amounts can be empirically determined and will vary with the particular condition being treated, the subject, and the efficacy and toxicity of each of the active agents contained in the composition. The actual dose to be administered will vary depending upon the age, weight, and general condition of the subject as well as the severity of the condition being treated, the judgment of the health care professional, and particular substituted pyrazolo[1,5-a]pyridine being administered.

Therapeutically effective amounts can be determined by those skilled in the art, and will be adjusted to the requirements of each particular case. Generally, a therapeutically effective amount of a substituted pyrazolo[1,5-a]pyridine of the invention will range from a total daily dosage of about 0.1 and 1000 mg/day, more preferably, in an amount between 1-200 mg/day, 30-200 mg/day, 1-100 mg/day, 30-100 mg/day, 30-300 mg/day, 1-60 mg/day, 1-40 mg/day, or 1-10 mg/day, administered as either a single dosage or as multiple dosages.

Preferred dosage amounts include dosages greater than or equal to about 10 mg BID, or greater than or equal to about 10 mg TID, or greater than or equal to about 10 mg QID. That is to say, a preferred dosage amount is greater than about 20 mg/day or greater than 30 mg/day. Dosage amounts may be selected from 30 mg/day, 40 mg/day, 50 mg/day, 60 mg/day, 70 mg/day, 80 mg/day, 90 mg/day or 100 mg/day or more. Depending upon the dosage amount and precise condition to be treated, administration can be one, two, or three times daily for a time course of one day to several days, weeks, months, and even years, and may even be for the life of the patient. Illustrative dosing regimes will last a period of at least about a week, from about 1-4 weeks, from 1-3 months, from 1-6 months, from 1-50 weeks, from 1-12 months, or longer.

Practically speaking, a unit dose of any given composition of the invention can be administered in a variety of dosing schedules, depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, every other day, three times weekly, twice weekly, once weekly, twice monthly, once monthly, and so forth.

### Formulations

In addition to comprising a substituted pyrazolo[1,5-a]pyridine of the invention, a therapeutic formulation of the invention may optionally contain one or more additional components as described below.

For example, a therapeutic composition may comprise, in addition to a substituted pyrazolo[1,5-a]pyridine, one or more pharmaceutically acceptable excipients or carriers. Exemplary excipients include, without limitation, polyethylene glycol (PEG), hydrogenated castor oil (HCO), cremophors, carbohydrates, starches (e.g., corn starch), inorganic salts, antimicrobial agents, antioxidants, binders/fillers, surfactants, lubricants (e.g., calcium or magnesium stearate), glidants such as talc, disintegrants, diluents, buffers, acids, bases, film coats, combinations thereof, and the like.

The amount of any individual excipient in the composition will vary depending on the role of the excipient, the dosage requirements of the active agent components, and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient. In general, the amount of excipient present in an composition comprising a substituted pyrazolo[1,5-a]pyridine is selected from at least about 2%, 5%, 10%, 15%, 20%, 25%, 30%, 35%, 40%, 45%, 50%, 55%, 60%, 65%, 70%, 75%, 80%, 85%, 90%, or even 95% by weight.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

A formulation (or kit) may contain, in addition to a substituted pyrazolo[1,5-a]pyridine, one or more additional active agents, e.g., a drug effective for treating neuropathic pain. Such actives include gabapentin, memantine, pregabalin, morphine and related opiates, cannabinoids, tramadol, lamotrigine, carbamazepine, duloxetine, milnacipran, and tricyclic antidepressants.

Preferably, the compositions are formulated in order to improve stability and extend the half-life of the active agent. For example, the substituted pyrazolo[1,5-a]pyridine may be delivered in a sustained-release formulation. Controlled or sustained-release formulations are prepared by incorporating the active into a carrier or vehicle such as liposomes, nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures. Additionally, a substituted pyrazolo[1,5-a]pyridine of the invention can be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996).

### Delivery Forms

The compositions described herein encompass all types of formulations, and in particular, those that are suited for systemic or intrathecal administration. Oral dosage forms include tablets, lozenges, capsules, syrups, oral suspensions, emulsions, granules, and pellets. Alternative formulations include aerosols, transdermal patches, gels, creams, ointments, suppositories, powders or lyophilates that can be reconstituted, as well as liquids. Examples of suitable diluents for reconstituting solid compositions, e.g., prior to injection, include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned. Preferably, a composition of the invention is one suited for oral administration.

Formulations suitable for parenteral administration include aqueous and non-aqueous isotonic sterile solutions suitable for injection, as well as aqueous and non-aqueous sterile suspensions. Parenteral formulations are optionally contained in unit-dose or multi-dose sealed containers, for example, ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the types previously described.

A formulation may also be a sustained release formulation, such that each of the drug components is released or absorbed slowly over time, when compared to a non-sustained release formulation. Sustained release formulations may employ pro-drug forms of the active agent, delayed-release drug delivery systems such as liposomes or polymer matrices, hydrogels, or covalent attachment of a polymer such as polyethylene glycol to the active agent.

In addition to the ingredients particularly mentioned above, the formulations of the invention may optionally include other agents conventional in the pharmaceutical arts and particular type of formulation being employed, for example, for oral administration forms, the composition for oral administration may also include additional agents as sweeteners, thickeners or flavoring agents.

The compositions of the present invention may also be prepared in a form suitable for veterinary applications.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

### EXAMPLES

The practice of the invention will employ, unless otherwise indicated, conventional techniques of organic synthesis, enzymatic assays, in-vitro and in-vivo models, and pharmacological evaluations, and the like, which are within the skill of the art. Such techniques are fully described in the literature. Reagents and materials are commercially available unless specifically stated to the contrary. See, for example, M. B. Smith and J. March, March's Advanced Organic Chemistry: Reactions Mechanisms and Structure, 6th Ed. (New York: Wiley-Interscience, 2007), supra, and Comprehensive Organic Functional Group Transformations II, Volumes 1-7, Second Ed.: A Comprehensive Review of the Synthetic Literature 1995-2003 (Organic Chemistry Series), Eds. Katritsky, A. R., et al., Elsevier Science, as well as technical references provided herein.

In the following examples, efforts have been made to ensure accuracy with respect to numbers used (e.g., amounts, temperatures, etc.) but some experimental error and deviation should be accounted for. Unless indicated otherwise, temperature is in degrees C. and pressure is at or near atmospheric pressure at sea level.

The following examples illustrate certain aspects and advantages of the present invention, however, the present invention is in no way considered to be limited to the particular embodiments described below.

### Example 1

### Synthesis of Substituted Pyrazolo[1,5-a]Pyridine Compounds

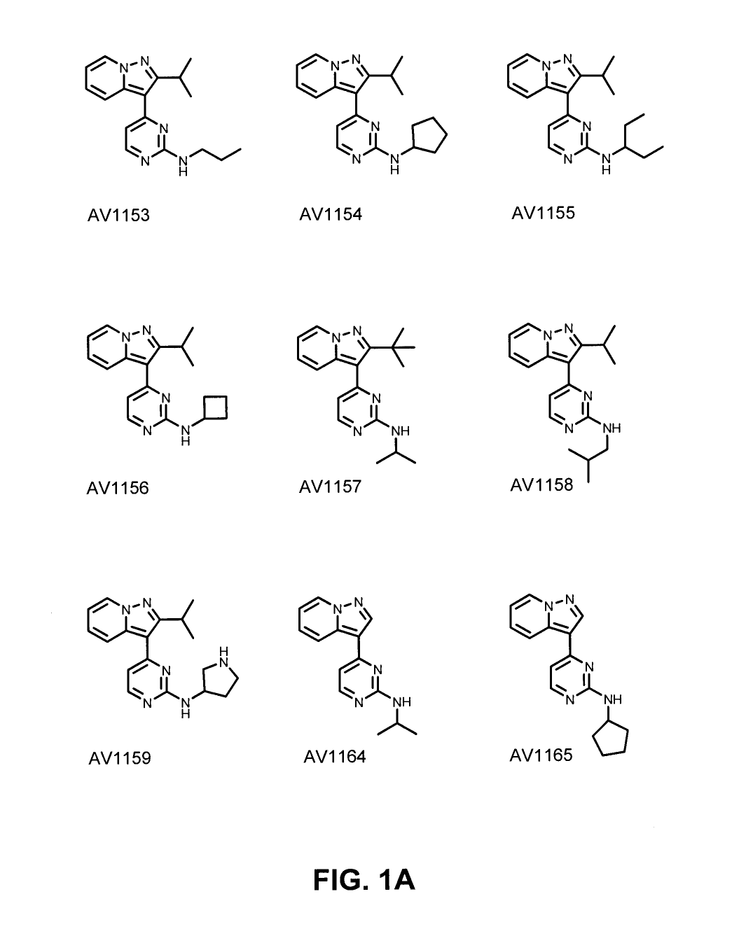

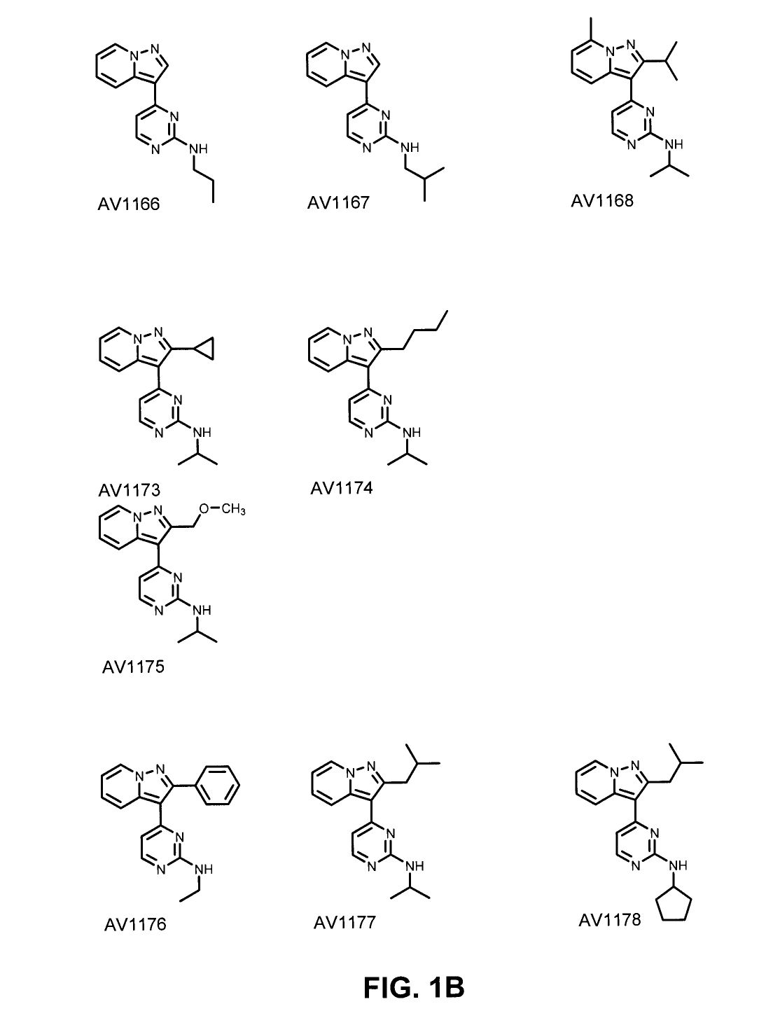

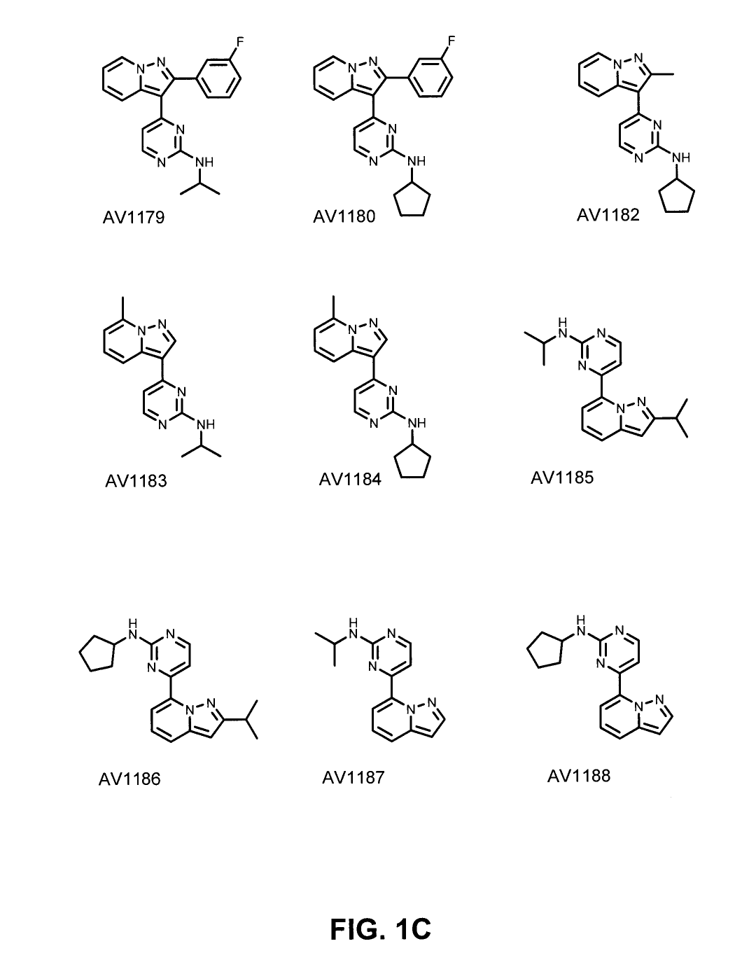

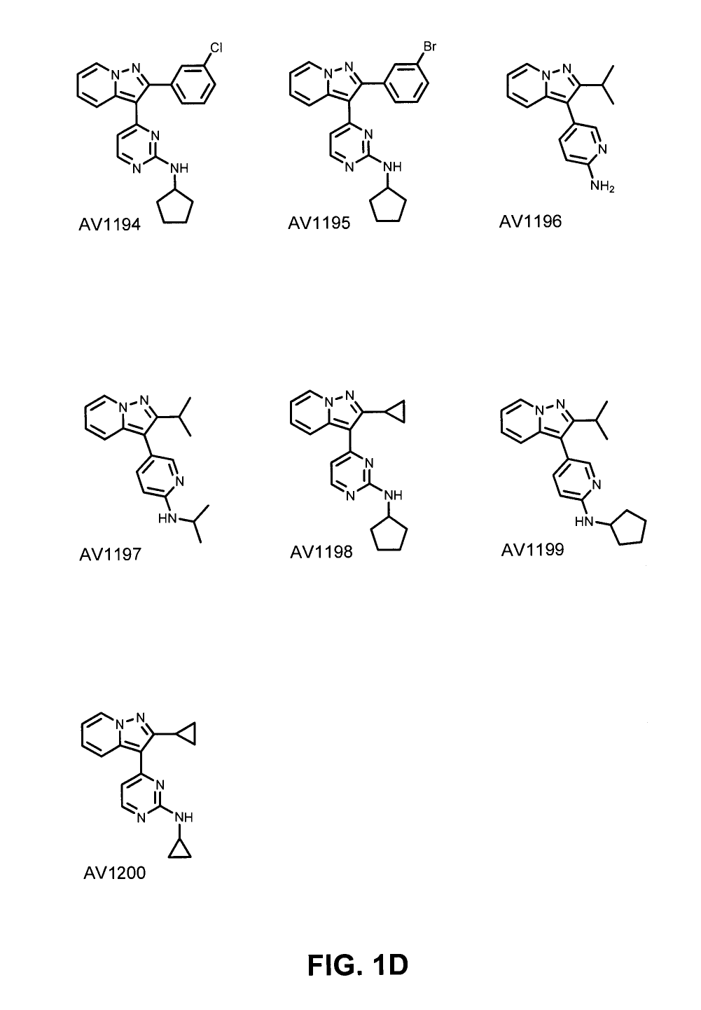

Compounds AV1153-1159, AV1164-1168, AV1173-1184, and AV1194-1200 were synthesized using conventional techniques of organic synthesis, purification, and characterization (e.g., IR, ¹H, ¹³C NMR, MS, elemental analysis), such as described in Applicant's U.S. Patent Application Publication No. 2008/0070912. Yields typically ranged from about 10% to about 90%, although focus was not on optimization of yields. Structures of certain of the compounds prepared, each a mono-, di- or tri-substituted pyrazolo[1,5-a]pyridine compound, are provided in FIGS. 1A-1D.

Illustrative syntheses of compounds AV 411, 1137, 1134, 1135, 1136, and 1139 are described in U.S. Patent Application Publication No. 2008/0070912.

SP600125, a reference JNK inhibitor, was obtained from Sigma. (Bennett, B. L., et al, SP600125, an anthrapyrazolone inhibitor of Jun N-terminal kinase. Proc. Natl. Acad. Sci. USA 98, 13681-13686, (2001))

TABLE 1
Compounds

| | | M + H (m/z) |
|---|---|---|
| | | Selected ¹H NMR |
| | | data [D₆-DMSO or |
| Compound | Structure | CDCl₃, δ (ppm)] |
| SP600125 | | |
| Papaverine | | |
| Rolipram | | |
| AV411 | | M + H 231 |
| 1-(2-isopropylpyrazolo[1,5- | | |
| a]pyridin-3-yl)-2-methylpropan-1- | | |
| one | | |
| AV1137 | | M + H 296; |
| N-isopropyl-4-(2- | | 8.56 (d, 1), 8.42 |
| isopropylpyrazolo[1,5-a]pyridin-3- | | (d, 1), 8.31 (d, 1), |
| yl)pyrimidin-2-amine | | 7.85 (t, 1), 7.52 |
| | | (t, 1), 7.05 (t, 1), |
| | | 6.93 (d, 1), 4.3 |
| | | (m, 1), 3.7 (m, 1), |
| | | 1.46 (d, 6), 1.41 |
| | | (d, 6) |
| AV1134 | | M + H 312; |
| 3-(4-(2-isopropylpyrazolo[1,5- | | 8.9 (d, 1), 8.3 (d, 1), |
| a]pyridin-3-yl)pyrimidin-2- | | 7.6 (t, 1), 7.2 (t, 1), |
| ylamino)propan-1-ol | | 7.1 (m, 1), 3.53 |
| | | (m, 2), 1.77 (m, 1), |
| | | 1.37 (d, 6) |
| AV1135 | | M + H 394; |
| 4-(2-isopropylpyrazolo[1,5- | | 8.71 (d, 1), 8.25 |
| a]pyridin-3-yl)-N-(3-(4- | | (d, 1), 7.37 (t, 1), |
| methylpiperazin-1- | | 7.11 (t, 1), 6.97 |
| yl)propyl)pyrimidin-2-amine | | (t, 1), 6.79 (d, 1), |
| | | 3.78 (m, 1), 2.22 |
| | | (m, 3), 1.72 (m, 2) |
| | | 1.35 (d, 6) |
| AV1136 | | M + H 294; |
| N-cyclopropyl-4-(2- | | 8.63 (d, 1), 8.56 |
| isopropylpyrazolo[1,5-a]pyridin-3- | | (d, 1), 7.87 (m, 1), |
| yl)pyrimidin-2-amine | | 7.52 (t, 1), 7.06 |
| | | (t, 1), 7.01 (d, 1), |
| | | 3.69 (m, 1), 2.93 |
| | | (m, 1), 1.47 (d, 6), |
| | | 0.96 (m, 2), 0.85 |
| | | m(2) |
| AV1139 | | M + H 254; |
| 4-(2-isopropylpyrazolo[1,5- | | 8.71 (d, 1), 8.33 |
| a]pyridin-3-yl)pyrimidin-2-amine | | (d, 1), 8.22 (d, 1), |
| | | 7.36 (m, 1), 6.96 |
| | | (m, 1), 6.79 (d, 1), |
| | | 6.55 (s, 2), 3.71 |
| | | (m, 1), 1.34 (d, 6) |
| AV1153 | | M + H 296; |
| 4-(2-isopropylpyrazolo[1,5- | | 8.88 (d, 1), 7.64 |
| a]pyridin-3-yl)-N-propylpyrimidin-2- | | (t, 1), 7.19 (t, 1), |
| amine | | 1.64 (m, 2), 1.37 |
| | | (d, 6), 0.96 (t, 3) |
| AV1154 | | M + H 322; |
| N-cyclopentyl-4-(2- | | 8.88 (d, 1), 8.79 |
| isopropylpyrazolo[1,5-a]pyridin-3- | | (m, 1), 8.30 (m, 1), |
| yl)pyrimidin-2-amine | | 7.64 (t, 1), 7.18 |
| | | (t, 1), 4.35 (m, 1), |
| | | 1.99 (m2), 1.72 |
| | | (m, 2), 1.63 (m, 4), |
| | | 1.37 (d, 6) |
| AV1155 | | M + H 324; |
| 4-(2-isopropylpyrazolo[1,5- | | 8.42 (d, 1), 8.22 |
| a]pyridin-3-yl)-N-(pentan-3- | | (d, 1), 7.22 (m, 1), |
| yl)pyrimidin-2-amine | | 6.78 (m, 1), 6.74 |
| | | (m, 1), 4.96 (m, 1), |
| | | 3.94 (m, 1), 3.71 |
| | | (m, 1), 1.58 (m, 4), |
| | | 1.41 (d, 6), 0.94 |
| | | (t, 6) |
| AV1156 | | M + H 308; |
| N-cyclobutyl-4-(2- | | 8.87 (d, 1), 8.25 |
| isopropylpyrazolo[1,5-a]pyridin-3- | | (m, 1), 7.645 (t, 1), |
| yl)pyrimidin-2-amine | | 7.16 (m, 1), 4.45 |
| | | (m, 1), 2.40 (m, 2), |
| | | 2.09 (m, 2), 1.77 |
| | | (m, 2), 1.37 (d, 6) |
| AV1157 | | M + H 310; |
| 4-(2-tert-butylpyrazolo[1,5-a]pyridin- | | 8.68 (d, 1), 8.28 |
| 3-yl)-N-isopropylpyrimidin-2-amine | | (d, 1), 7.51 (d, 1), |
| | | 7.25 (t, 1), 6.88 |
| | | (t, 1), 6.64 (d, 1), |
| | | 4.10 (m, 1), 1.42 |
| | | (s, 9), 1.16 (d, 6) |
| AV1158 | | M + H 310; |
| N-isobutyl-4-(2- | | 8.71 (d, 1), 8.23 |
| isopropylpyrazolo[1,5-a]pyridin-3- | | (d, 1), 7.37 (t, 1), |
| yl)pyrimidin-2-amine | | 7.15 (t, 1), 6.97 |
| | | (t, 1), 6.78 (d, 1), |
| | | 3.79 (m, 1), 3.15 |
| | | (t, 2), 1.90 (m, 1), |
| | | 1.34 (d, 6), 0.91 |
| | | (d, 6) |
| AV1159 | | M + H 323; |
| 4-(2-isopropylpyrazolo[1,5- | | 8.78 (d, 1), 8.31 |
| a]pyridin-3-yl)-N-(pyrrolidin-3- | | (m, 1),, 8.22 (m, 1), |
| yl)pyrimidin-2-amine | | 7.65 (t, 1), 7.21 |
| | | (m, 2), 4.68 (m, 1), |
| | | 3.76 (m, 1), 3.58 |
| | | (m, 1), 3.37 (m, 4), |
| | | 2.36 (m, 1), 2.11 |
| | | (m, 1), 1.34 (d, 6) |
| AV1164 | | M + H 254; |
| N-isopropyl-4-(pyrazolo[1,5- | | 8.99 (s, 1), 8.93 |
| a]pyridin-3-yl)pyrimidin-2-amine | | (d, 1), 8.52 (m, 1), |
| | | 8.24 (m, 1), 7.69 |
| | | (t, 1), 7.33 (d, 1), |
| | | 7.24 (t, 1), 1.29 |
| | | (d,6) |
| AV1165 | | M + H 280; |
| N-cyclopentyl-4-(pyrazolo[1,5- | | 8.99 (s, 1), 8.94 |
| a]pyridin-3-yl)pyrimidin-2-amine | | (d, 2), 8.80 (m, 1), |
| | | 8.56 (m, 1), 8.24 |
| | | (d, 1), 7.68 (t, 1), |
| | | 7.34 (d, 1), 7.21 |
| | | (t, 1), 2.04 (m, 2), |
| | | 1.71 (m, 7) |
| AV1166 | | M + H 254; |
| N-propyl-4-(pyrazolo[1,5-a]pyridin- | | 8.99 (s, 1), 8.94 |
| 3-yl)pyrimidin-2-amine | | (d, 1), 8.56 (m, 1), |
| | | 8.24 (d, 1), 7.69 |
| | | (m, 1), 7.33 (d, 1), |
| | | 7.24 (t, 1), 3.47 |
| | | (m, 2), 1.65 (m, 2), |
| | | 0.98 (t, 3) |
| AV1167 | | M + H 268; |
| N-isobutyl-4-(pyrazolo[1,5-a]pyridin- | | 9.00 (s, 1), 8.94 |
| 3-yl)pyrimidin-2-amine | | (d, 1), 8.77 (m, 1), |
| | | 8.49 (m, 1), 8.24 |
| | | (m, 1), 7.69 (m, 1), |
| | | 7.34 (d, 1), 7.24 |
| | | (t, 1), 3.35 (m, 1), |
| | | 3.16 (m, 1), 1.96 |
| | | (m, 1), 0.97 (d, 3) |
| AV1168 | | M + H 310; |
| N-isopropyl-4-(2-isopropyl-7- | | 8.67 (m, 1), 8.26 |
| methylpyrazolo[1,5-a]pyridin-3- | | (m, 2), 7.61 (t, 1), |
| yl)pyrimidin-2-amine | | 7.16 (m, 2), 4.25 |
| | | (m, 1), 3.82 (m, 1), |
| | | 2.75 (s, 3), 1.40 |
| | | (d, 6), 1.30 (2, 6) |
| AV1173 | | M + H 294; |
| 4-(2-cyclopropylpyrazolo[1,5- | | 14.6 (s, 1), 8.48 |
| a]pyridin-3-yl)-N-isopropylpyrimidin- | | (d, 1), 8.44 (d, 1), |
| 2-amine | | 7.84 (t, 1), 7.53 |
| | | (t, 1), 7.40 (d, 2), |
| | | 7.05 (t, 1), 4.35 |
| | | (m, 1), 2.24 (m, 1), |
| | | 1.73 (m, 2), 1.43 |
| | | (d, 6), 1.97 (m, 4) |
| AV1174 | | M + H 310; |
| 4-(2-butylpyrazolo[1,5-a]pyridin-3- | | 8.69 (d, 1), 8.24 |
| yl)-N-isopropylpyrimidin-2-amine | | (d, 1), 7.39 (t, 1), |
| | | 6.96 (m, 2), 6.77 |
| | | (d, 1), 4.12 (m, 1), |
| | | 3.06 (m, 2), 1.70 |
| | | (m, 2), 1.37 (m, 2), |
| | | 1.19 (d, 6), 0.91 |
| | | (t, 3) |
| AV1175 | | M + H 298; |
| N-isopropyl-4-(2- | | 8.77 (d, 1), 8.6 |
| (methoxymethyl)pyrazolo[1,5- | | (m, 1), 8.24 (d, 1), |
| a]pyridin-3-yl)pyrimidin-2-amine | | 7.45 (t, 1), 7.06 |
| | | (t, 1), 6.93 (m, 2), |
| | | 4.76 (s, 2), 4.10 |
| | | (m, 1), 3.34 (s, 3), |
| | | 1.20 (d, 6) |
| AV1176 | | M + H 330; |
| N-isopropyl-4-(2- | | 8.80 (d, 1), 8.6 |
| phenylpyrazolo[1,5-a]pyridin-3- | | (m, 1), 8.01 (d, 1), |
| yl)pyrimidin-2-amine | | 7.5 (m, 6), 7.06 |
| | | (m, 2), 6.15 (d, 1), |
| | | 4.07 (m, 1), 1.18 |
| | | (d, 6) |
| AV1177 | | M + H 310; |
| 4-(2-isobutylpyrazolo[1,5-a]pyridin- | | 8.89 (d, 1), 8.67 |
| 3-yl)-N-isopropylpyrimidin-2-amine | | (m, 1), 8.28 (m, 1), |
| | | 7.66 (m, 1), 7.20 |
| | | (t, 1), 7.13 (m, 1), |
| | | 4.3 (m, 1), 3.01 |
| | | (d, 2), 2.13 (m, 1), |
| | | 1.30 (d, 6), 0.95 |
| | | (d,6) |
| AV1178 | | M + H 336; |
| N-cyclopentyl-4-(2- | | 8.89 (d, 1), 8.42 |
| isobutylpyrazolo[1,5-a]pyridin-3- | | (m, 1), 8.31 (m, 1), |
| yl)pyrimidin-2-amine | | 7.65 (m, 1), 7.20 |
| | | (t, 1), 7.11 (m, 1), |
| | | 4.2 (m, 1), 3.01 |
| | | (d, 2), 2.03 (m, 3), |
| | | 1.7 (m, 6), 0.95 |
| | | (d,6) |
| AV1179 | | M + H 348; |
| 4-(2-(3-fluorophenyl)pyrazolo[1,5- | | 9.1 (m, 1), 8.97 |
| a]pyridin-3-yl)-N-isopropylpyrimidin- | | (d, 1), 8.7 (m, 1), |
| 2-amine | | 8.5 (m, 1), 8.1 |
| | | (m, 2), 7.7-7.2 |
| | | (m, 6), 6.5-6.3 |
| | | (m, 1), 4.16 (m, 1), |
| | | 1.26 (d, 6) |
| AV1180 | | M + H 374; |
| N-cyclopentyl-4-(2-(3- | | 9.1 (m, 1), 8.97 |
| fluorophenyl)pyrazolo[1,5-a]pyridin- | | (d, 1), 8.82 (m, 1), |
| 3-yl)pyrimidin-2-amine | | 8.5 (m, 1), 8.1 |
| | | (m, 1), 7.8 (m, 1), |
| | | 7.6-7.4 (m, 4), 7.31 |
| | | (t, 1), 6.5-6.3 (m, 1), |
| | | 4.2 (m, 1), 1.9 |
| | | (m, 2), 1.75-1.5 |
| | | (m, 6) |
| AV1182 | | M + H 294; |
| N-cyclopentyl-4-(2- | | 8.87 (m, 2), 8.5 |
| methylpyrazolo[1,5-a]pyridin-3- | | (m, 1), 8.3 (m, 1). |
| yl)pyrimidin-2-amine | | 7.68 (m, 1), 7.2 |
| | | (d, 1), 7.17 (m, 1), |
| | | 4.4-4.2 (m, 1), 2.71 |
| | | (s, 3), 2.03 (m, 2), |
| | | 1.8-1.6 (m, 6) |
| AV1183 | | M + H 268; |
| N-isopropyl-4-(7- | | 9.09 (s, 1), 8.8 |
| methylpyrazolo[1,5-a]pyridin-3- | | (m, 1), 8.4-8.2 |
| yl)pyrimidin-2-amine | | (m, 2), 7.7 (m, 1), |
| | | 7.4 (d, 1), 7.2 (d, 1), |
| | | 4.3 (m, 1), 2.77 |
| | | (s, 3), 1.27 (d, 6) |
| AV1184 | | M + H 294; |
| N-cyclopentyl-4-(7- | | 9.09 (s, 1), 8.85 |
| methylpyrazolo[1,5-a]pyridin-3- | | (m, 1), 8.5-8.2 |
| yl)pyrimidin-2-amine | | (m, 2), 7.7 (m, 1), |
| | | 7.42 (d, 1), 7.21 |
| | | (d, 1), 4.5-4.1 |
| | | (m, 1), 2.7(m, 1), |
| | | 2.78 (s, 3), 2.0 |
| | | (m, 2), 1.6 (m,6) |
| AV1194 | | M + H 390; |
| 4-(2-(3-chlorophenyl)pyrazolo[1,5- | | 8.99 (m, 3), 8.2 |
| a]pyridin-3-yl)-N- | | (m, 2), 7.8-7.5 |
| cyclopentylpyrimidin-2-amine | | (m, 5), 7.32 (t, 1), |
| | | 6.6-6.4 (m, 1), 4.2- |
| | | 4.1 (m, 1), 3.76 |
| | | (m, 1), 1.9-1.5 |
| | | (m, 8) |
| AV1195 | | M + H 434; |
| 4-(2-(3-bromophenyl)pyrazolo[1,5- | | 8.96 (2, 1), 8.8 |
| a]pyridin-3-yl)-N- | | (m, 1), 8.1 (m, 1), |
| cyclopentylpyrimidin-2-amine | | 7.8 (m, 1), 7.7 |
| | | (m, 2), 7.6 (m, 1), |
| | | 7.5 (m, 1), 7.3 (t, 1), |
| | | 6.5-6.3 (m, 1), 4.1 |
| | | (m, 1), 1.97 (m, 2), |
| | | 1.8-1.5 (m, 6) |
| AV1196 | | M + H 253 |
| 5-(2-isopropylpyrazolo[1,5- | | |
| a]pyridin-3-yl)pyridin-2-amine | | |
| AV1197 | | M + H 295; |
| N-isopropyl-5-(2- | | |
| isopropylpyrazolo[1,5-a]pyridin-3- | | |
| yl)pyridin-2-amine | | |
| AV1198 | | M + H 320; |
| N-cyclopentyl-4-(2- | | 9.0 (m, 1), 8.82 |
| cyclopropylpyrazolo[1,5-a]pyridin-3- | | (d, 1), 8.3 (m, 2), |
| yl)pyrimidin-2-amine | | 7.8 (m, 1), 7.47 |
| | | (d, 1), 7.18 (t, 1), |
| | | 4.3 (m, 1), 2.1 |
| | | (m, 2), 1.7 (m, 6), |
| | | 1.1 (m, 4) |
| AV1199 | | M + H 321; |
| N-cyclopentyl-5-(2- | | 8.8(m, 1), 8.69 |
| isopropylpyrazolo[1,5-a]pyridin-3- | | (d, 1), 7.93 (d, 2), |
| yl)pyridin-2-amine | | 7.53 (d, 1), 7.25 |
| | | (t, 1), 7.11 (d, 1), |
| | | 6.91 (t, 1), 4.1 |
| | | (m, 1), 3.16 (m, 1), |
| | | 2.0 (m, 2), 1.8-1.6 |
| | | (m, 6), 1.29 (d, 6) |
| AV1200 | | M + H 292; |
| N-cyclopropyl-4-(2- | | 14.7 (s, 1), 8.79 |
| cyclopropylpyrazolo[1,5-a]pyridin-3- | | (d, 1), 8.63 (s, 1), |
| yl)pyrimidin-2-amine | | 8.48 (d, 1), 7.86 |
| | | (m, 1), 7.52 (t, 1), |
| | | 7.48 (d, 1), 7.06 |
| | | (t, 1), 2.95 (m, 1), |
| | | 2.25 (m, 1), 1.21 |
| | | (m, 4), 0.97 (m, 2), |
| | | 0.85 (m, 2) |
| AV1185 N-isopropyl-4-(2- isopropylpyrazolo[1,5-a]pyridin-7- yl)pyrimidin-2-amine | 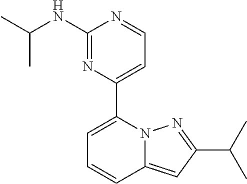 | |
| AV1186 N-cyclopentyl-4-(2- isopropylpyrazolo[1,5-a]pyridin-7- yl)pyrimidin-2-amine | 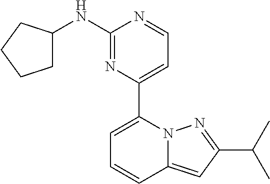 | |
| AV1187 N-isopropyl-4-(pyrazolo[1,5- a]pyridin-7-yl)pyrimidin-2-amine | 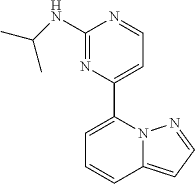 | |
| AV1188 N-cyclopentyl-4-(pyrazolo[1,5- a]pyridin-7-yl)pyrimidin-2-amine | 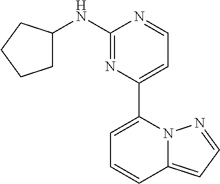 | |
| AV1189 N-4-(2-isopropylpyrazolo[1,5- a]pyridin-3-yl)pyrimidin-2- yl)methanesulfonamide | 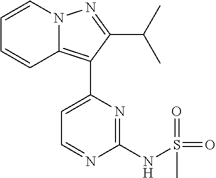 | |
| AV1190 N-4-(2-isopropylpyrazolo[1,5- a]pyridin-3-yl)pyrimidin-2- yl)benzenesulfonamide | 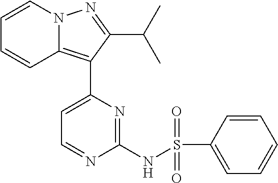 | |
| AV1191 N-4-(2-methylpyrazolo[1,5- a]pyridin-3-yl)pyrimidin-2- yl)thiphene-3-sulfonamide | 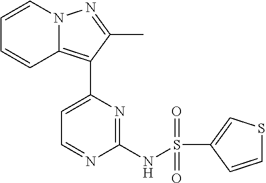 | |
| AV1192 N-4-(2-isopropylpyrazolo[1,5- a]pyridin-3-yl)pyrimidin-2- yl)quinoline-8-sulfonamide | 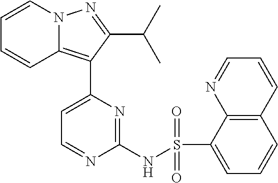 | |
| AV1193 N-4-(pyrazolo[1,5-a]pyridin-3- yl)pyrimidin-2-yl) benzenesulfonamide | 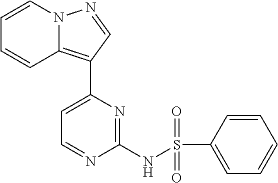 | |
| AV1194 4-(2-(3-chlorophenyl)pyrazolo[1,5- a]pyridin-3-yl)-N- cyclopentylpyrimidin-2-amine | 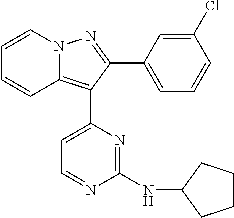 | |
| AV1201 N-cyclopentyl-4-(-2- isopropylpyrazolo[1,5-a]pyridin-3- yl)pyridin-2-amine | 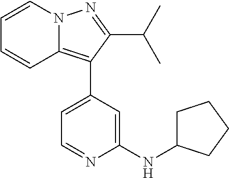 | |
| AV1202 N-isopropyl-4-(-2- isopropylpyrazolo[1,5-a]pyridin-3- yl)pyridin-2-amine | 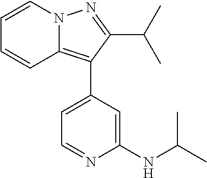 | |
| AV1203 5-(-2-isopropylpyrazolo[1,5- a]pyridin-3-yl)pyrimidin-2-amine | 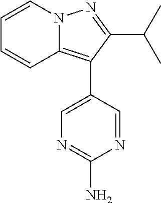 | |
| AV1205 4-(-2-isopropylpyrazolo[1,5- a]pyridin-3-yl)pyridin-2-amine | 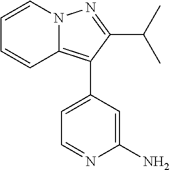 | |
| AV1206 N-isopropyl-5-(-2- isopropylpyrazolo[1,5-a]pyridin-3- yl)pyrimidin-2-amine | 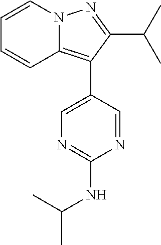 | |
| AV1207 N-cyclopentyl-5-(-2- isopropylpyrazolo[1,5-a]pyridin-3- yl)pyrimidin-2-amine | 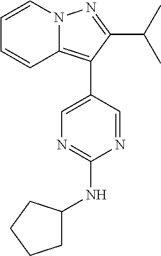 | |
| AV1208 4-(-2-isopropylpyrazolo[1,5- a]pyridin-3-yl)-N,N-dimethylpyridin- 2-amine | 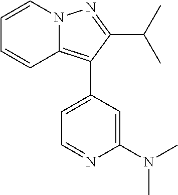 | |

### Example 2

### In-Vitro Enzymatic Assays

The biological activities of the subject compounds were evaluated using the enzymatic and cell-based assays described below. The assays described utilize Parkinson's and Alzheimer's disease related toxins to model disease state.

BV-2 Glial cell Assay

Mouse BV-2 microglial cells were seeded on 96-well plates at a concentration of 3×10⁴ cells/well. Cells were activated with 100 ng/mL LPS and IFN-γ (100 ng/mL) in the presence or absence of test compounds (0-30 uM). Following incubation for 20 hours, cells were spun down and supernatant collected. The supernatant was analyzed via Luminex for the presence of TNF-α and MCP-1.

Phosphorylated c-JUN (6-OHDA)

Human SH-SY5Y neuroblastoma cells were cultured in 96-well plates with 2.5×10⁴ cells/well and incubated for 48 hours. 6-hydroxydopamine was added at a concentration of 50 uM in the presence or absence of test compounds (0-30 uM) and incubated for 1.5 hours. Supernatant was discarded and cells were fixed with 4% paraformaldehyde (100 ul/well) for 20 minutes. FACE cell based ELISA assay (Active Motif) was performed specific for quantifying phosphorlyated c-Jun (serine-73).

Phosphorylated c-JUN (A-beta)

In 96-well plates either human SH-SY5Y neuroblastoma cells at 2.5×10⁴ cells/well or E18 rat neuronal cells at 1.5×10⁴ cells/well were seeded. To each well the following was added, Amyloid Beta peptide 1-25 (source) at a concentration of 30 uM, retinoic acid (10 uM) and test compounds (0-30 uM) and incubated for 3 hours. Supernatant was discarded and cells were fixed with 4% paraformaldehyde (100 ul/well) for 20 minutes. FACE cell based ELISA assay (Active Motif) was performed specific for quantifying phosphorlyated c-Jun (serine-73).

### JNK Inhibition Assays:

In a 96-well plate, recombinant full length human JNK2 or JNK3 enzyme (40 ng) expressed in Sf21 cells (Upstate) was incubated with the substrate ATF2 (3 uM) for 1 hour in the presence of 10 uM ATP. The amount of ATP depletion by the JNK enzyme is measured in the presence or absence of test compounds (0-30 uM). The ATP levels were quantified using luciferase with luminescence read on a Victor Light 1420 luminometer. IC₅₀ calculations were plotted using a nonlinear regression curve fit.

### PDE4 Inhibition Assays:

In a 96-well plate, the catalytic domain of phosphodiesterase 4B enzyme (40 mU/well) cloned from human brain (Scottish Biomedical) was combined with 5 uM cAMP substrate (Sigma). Test compounds (0-30 uM) or vehicle (0.5% DMSO) were added to the enzyme/substrate and incubated 1 hour. Using a PDELight® kit (Cambrex), the amount of AMP produced in the reaction from the hydrolysis of cAMP was quantified using PDELight AMP Detection Reagent which converts the AMP directly to ATP. The assay uses luciferase, which catalyses the formation of light from the newly formed ATP and luciferin. The luminescence was read on a Victor Light 1420 luminometer. IC₅₀ calculations were plotted using a nonlinear regression curve fit.

### PDE10 Inhibition Assay

In a 96-well plate, recombinant human phosphodiesterase 10A1 enzyme (10 U/well) expressed in Baculovirus infected Sf9 cells (BPS Biosciences) was combined with 1 uM cAMP substrate (Sigma). Test compounds (0-30 uM) or vehicle (0.5% DMSO) were added to the enzyme/substrate and incubated 1 hour. Using a PDELight® kit (Cambrex), the amount of AMP produced in the reaction from the hydrolysis of cAMP was quantified using PDELight AMP Detection Reagent which converts the AMP directly to ATP. The assay uses luciferase, which catalyses the formation of light from the newly formed ATP and luciferin. The luminescence was read on a Victor Light 1420 luminometer. IC₅₀ calculations were plotted using a nonlinear regression curve fit.

Assay results are summarized in Tables 2 and 3. Papavarine is a non-selective PDE inhibitor, and rolipram is a known specific PDE-4 inhibitor; IC50 values are provided as a basis for comparison.

TABLE 2

| | JNK 3 | JNK 2 | PDE 10 | PDE 4 |
|---|---|---|---|---|
| | Inhibition | Inhibition | Inhibition | Inhibition |
| Compound | (IC50 in μM) | (IC50 in μM) | (IC50 in μM) | (IC50 in μM) |
| SP600125 | 0.2 | 0.2 | N/A | N/A |
| Papaverine | N/A | N/A | 0.3-3.7 | N/A |
| Rolipram | N/A | N/A | N/A | 3.4 |
| AV411 | >100 | >100 | 6.3 | 7.3 |
| AV1137 | 2.6 | 1.6 | 9.9 | 4.3 |
| AV1134 | 8.2 | | 9.3 | 6.2 |
| AV1135 | 83 | | 19 | >100 |
| AV1136 | 1.5 | | 16.8 | 4.5 |
| AV1139 | 18 | | >100 | >100 |
| AV1153 | 4.1 | | 8.9 | 11.5 |
| AV1154 | 6.0 | | 3.0 | 7.1 |
| AV1155 | 27 | | 42 | 5.9 |
| AV1156 | 3.0 | | 20 | 8.6 |
| AV1157 | >100 | | >100 | >100 |
| AV1158 | 1.2 | | 8.4 | 3.4 |
| AV1164 | 0.3 | 0.5 | 5.8 | 5.9 |
| AV1165 | 0.2 | 0.4 | 7.5 | 14 |
| AV1166 | 0.4 | 1.3 | 38.2 | 16.5 |
| AV1167 | 0.7 | 1.5 | 37 | 13 |
| AV1168 | 2.2 | 8.1 | 59 | 2.9 |
| AV1173 | 1.2 | 0.3 | 7.4 | 1.1 |
| AV1174 | 1.1 | 1.2 | 31 | 7.9 |
| AV1175 | 1.2 | 4.5 | >100 | 8.6 |
| AV1176 | 0.1 | 0.1 | >100 | 3.8 |
| AV1177 | 1.8 | 3.0 | >100 | 6.4 |
| AV1178 | 3.6 | 2.8 | >100 | 5.4 |
| AV1179 | 0.2 | | >100 | >100 |
| AV1180 | 0.3 | | 53.5 | 16.4 |
| AV1182 | 0.9 | | >100 | 5.7 |
| AV1183 | 0.2 | | 35 | 6.2 |
| AV1184 | 0.4 | | 32.0 | 15.5 |
| AV1194 | 0.08 | 0.1 | >100 | 5.2 |
| AV1195 | 0.05 | 0.09 | >100 | 4.9 |
| AV1196 | 68 | | 8.3 | 20.8 |
| AV1197 | >100 | | 17 | 50.5 |
| AV1198 | 2.8 | 1.4 | 6.8 | 3.4 |
| AV1199 | >100 | | 7.1 | 43 |
| AV1200 | 1.9 | 1.6 | 7.7 | 2.7 |
| AV1185 | >100 | | >100 | 57 |
| AV1186 | >100 | | >100 | nd |
| AV1187 | >100 | | >100 | nd |
| AV1188 | >100 | | >100 | nd |
| AV1189 | >100 | | >100 | nd |
| AV1190 | >100 | | nd | 40 |
| AV1191 | >100 | | >100 | 6.8 |
| AV1192 | nd | | nd | nd |
| AV1193 | 53 | | >100 | 9.4 |
| AV1194 | 0.08 | 0.1 | >100 | 5.1 |
| AV1021 | >100 | | 12 | >100 |
| AV1202 | >100 | | 13 | >100 |
| AV1203 | >10 | >100 | 3.8 | 9.9 |
| Av1204 | >100 | | 65 | 61 |
| AV105 | >100 | | >100 | 22 |
| AV1206 | >100 | | 7.4 | 43 |
| AV1207 | >100 | | 3.2 | 24 |
| AV1208 | >100 | | 5.4 | 3.9 |

TABLE 3

| | | | p-cJun | p-cJun | p-cJun |
|---|---|---|---|---|---|
| | MCP-1 | TNF-a | 6-OHDA | Abeta | Abeta |
| | BV-2 | BV-2 | SH-SY5Y | SH-SY5Y | E18 |
| | (EC50 in | (EC50 in | (EC50 in | (EC50 in | (EC50 in |
| Compound | μM) | μM) | μM) | μM) | μM) |
| SP600125 | | | 0.6 | | 7.6 |
| AV411 | 42 | 2.7 | >30 | 3.8 | >30 |
| AV1137 | 1.1 | 1.2 | 6.1 | >30 | 3.0 |
| AV1153 | | | >30 | >30 | 3.6 |
| AV1154 | | | 3.4 | >10 | 13 |
| AV1158 | 1.2 | 1.0 | >30 | >30 | |
| AV1164 | 4.9 | 0.4 | 3.1 | 2.9 (no | 1.4 |
| | | | | Abeta | |
| | | | | 1.1) | |
| AV1165 | 0.6 | 0.2 | 1.1 | 2.4 (no | 15 |
| | | | | Abeta | |
| | | | | 7.6) | |
| AV1166 | 1.1 | 0.9 | 1.4 | 11 | 37 |
| AV1173 | 0.7 | 1.4 | 7.7 | | |
| AV1178 | | | | >30 | |
| AV1180 | 1.6 | 0.04 | >30 | 4.1 | |
| AV1183 | 1.2 | 1.1 | 0.3 | 5.4 | |
| AV1184 | 0.9 | 0.3 | 0.7 | 8.3 | |
| AV1194 | 1.2 | 0.03 | | | |
| AV1195 | 0.7 | 0.03 | | | |
| AV1198 | 0.3 | 3.3 | | | |
| AV1200 | 0.3 | 1.0 | | | |
| AV1203 | 0.3 | 0.8 | | | |

The enzymatic assay results above indicate that the subject compounds exhibit activity against PDE 4, PDE10, and JNK kinases (2 &3). This unique multi-target activity suggests that these compounds may have utility in multiple indications including neurodegenerative diseases (e.g. Parkinson's and Alzheimer's) in addition to treatment of neuropathic pain. Moreover, the subject compounds are capable of regulating glial cell activation, as demonstrated by their ability to inhibit cytokines in glial cell lines (see, e.g., MCP-1 BV-2 EC50 and TNF-a BV-2 EC50 data in Table 3).

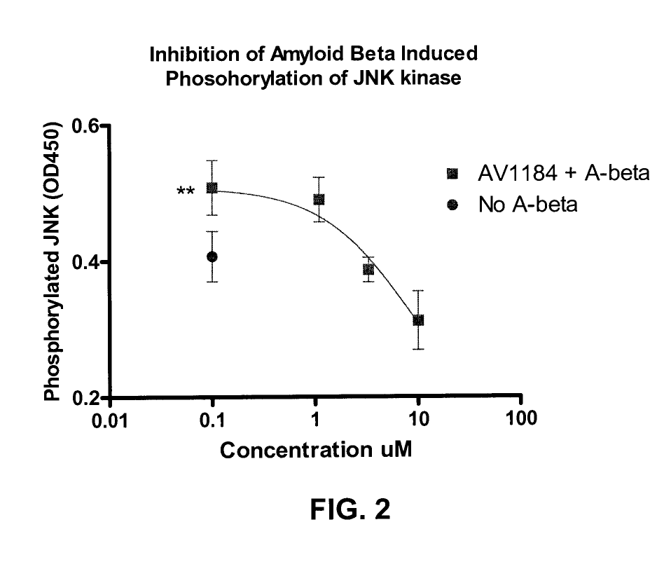

A representative example for AD (Alzheimer's disease) is shown in FIG. 2. Neuronal cells were stimulated with Amyloid Beta, the Alzheimer's related peptide, resulting in phosphorylation of JNK kinase as measured by ELISA. As can be seen from the graphical results, AV1184 is capable of inhibiting phosphorylation of JNK kinase in a dose-dependent manner, indicating its potential efficacy in treating AD among other features.

### Example 3

### Pharmacological Evaluation

### Neurodegenerative Indication in Parkinson's Model

### 6-OHDA (Parkinson's) Model

AV1173 was evaluated in a 6-OHDA lesioned rats, a standard rat model of Parkinson's disease. (See, e.g., Ungerstedt, U. “6-hydroxydopamine induced degeneration of monoamine neurons.” Eur. J. Pharm. 5: 107-110, 1968; Garvey P M, et al., “Injection of biologically relevant active substances into the brain.” Methods in Neurosciences. 21: 214-234, 1994).

Pre-lesioned rats were purchased from a vendor (Taconic) following stereotaxic injection into the brain of the neurotoxin 6-hydroxydopamine (6-OHDA), which leads to neuronal cell loss in those brain regions affected in PD (in particular the substantia nigra). Briefly, anesthetized rats were injected with 5 mg of 6-OHDA using stereotaxic coordinates to locate the needle within the nigrostatial pathway. A 2 ug/uL solution of 6-OHDA was infused at a rate of 1 ul/min for four minutes. These 6-OHDA-treated rats exhibit a characteristic rotational behavior when treated with a dopamine-like compound (apomorphine, 0.5 mg/kg SC). On Day 10 post-surgery, a baseline rotational behavior was assessed over 45 minutes following an injection of 0.5 mg/kg SC apomorphine (Sigma) using a rotometer (RotoMax analyzer). On Day 11 post-injection, rats began a once-daily oral regimen of AV1173 (50 mg/kg PO). Rats were assessed for rotational behavior following 0.5 mg/kg apomorphine challenge, one week from the initiation of dosing (Day 17 post-6-OHDA injection).

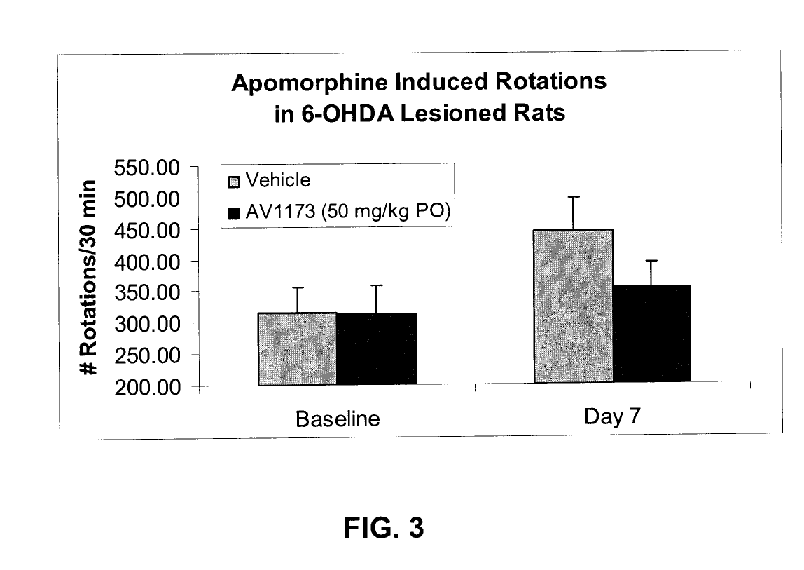

AV1173 treatment (50 mg/kg PO) for one week reduced the rotational behavior relative to rats dosed with vehicle control. See FIG. 3. After 7 days, the average number of apomorphine induced rotations in 30 minutes in the control group had increased from 315 (baseline) to 444, while in comparison, for the group treated with AV1173, the number of rotations observed was only 354. Thus, it can be seen that compound AV1173 is effective in reducing apomorphine induced rotational behavior in rats relative to rats dosed with vehicle control—indicating the potential usefulness of the subject compound in treatment of Parkinson's disease.

### Example 4

### Pharmacological Evaluation

### Cognitive Indications

The potential cognitive enhancing properties of the exemplary compound, AV1137, were examined in the Morris Maze test in the rat. The Morris Water Maze Test was carried out as described in Morris R. G. M., “Spatial localization does not require the presence of local cues.”, Learning and Motivation, 12, 239-260, 1981.

Male Wistar rats were given 4 training sessions over 4 consecutive days. The training session consisted of 4 consecutive trials in the Morris Maze, each separated by 60 seconds. For each trial, the animal was placed in the maze at one of two starting points equidistant from the escape platform and allowed to find the escape platform. The animal was left on the escape platform for 60 seconds before starting a new trial. If the animal did not find the platform within 120 seconds, the experimenter removed it from the water and placed it on the platform for 60 seconds. During the 4 trials the animals started the maze twice from each starting point in a randomly determined order per animal.

The trials were video-recorded and the behaviour of animals was analysed using a video-tracking system (Panlab: SMART). The measures taken were the escape latency, the path length and the swim speed at each trial. Scopolamine (0.5 mg/kg i.p.), administered 30 minutes before each session, induces amnesia as indicated by the failure of scopolamine-treated rats to reduce their escape latencies from trial to trial. 12 rats were studied per group. The test was performed blind.

AV1137 was evaluated at 2 and 5 mg/kg, administered twice daily. Compound was administered i.p. 60 minutes before each session. That is to say, compound was administered 30 minutes before scopolamine or alone at 5 mg/kg administered i.p. 60 minutes before each session (i.e. 30 minutes before an injection of physiological saline).

AV1137 was also administered at the end of each acquisition day (i.e. 6-8 hours after the first administration). The experiment included a normal control group (vehicle/saline) and an amnesic control group (vehicle/scopolamine) receiving the same number of administrations of vehicle. Each experiment therefore included 5 groups. Data were analyzed by comparing treated groups with appropriate control using unpaired Student's t tests.

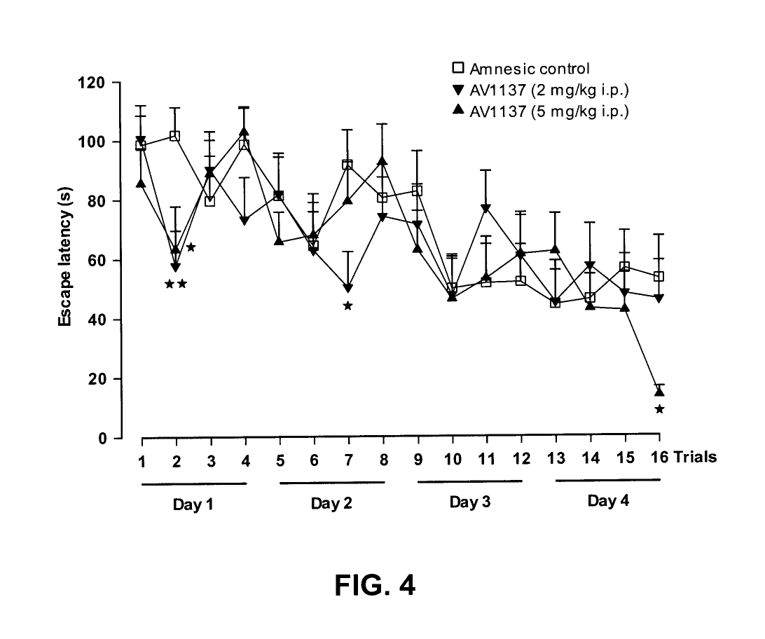

A trend was seen for decreased escape latency and distance swum in AV1137 treated rats relative to scopolamine treated controls indicating potential improvement in cognitive function following AV1137 treatment. See FIG. 4.

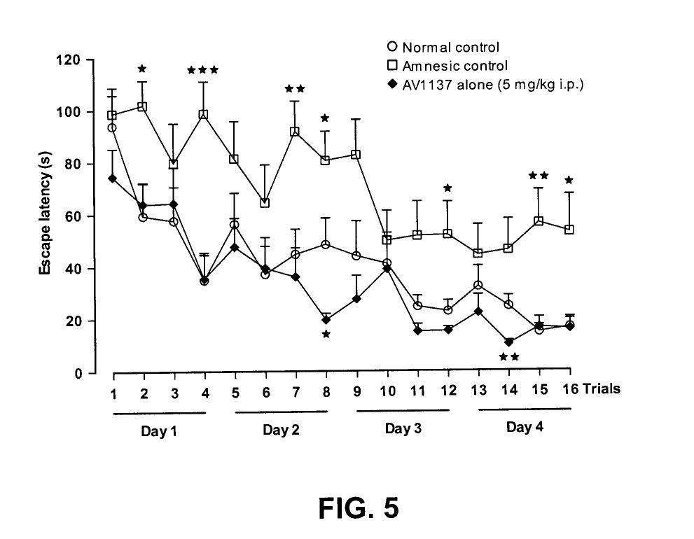

In addition, AV1137 treatment produced a trend for reduced escape latencies in normal rats (no scopolamine treatment), relative to saline controls. See FIG. 5. These combined results indicate that AV1137 may possess cognitive-enhancing effects. 4 trials were carried out each day, each data-point is represented on the x-axis)*=p<0.05; **=p<0.01; ***=p<0.001.

### Example 5

### Pharmacological Evaluation

### Neuropathic Pain

Compounds described herein were evaluated in a standard rat chronic constriction injury (CCI) model of neuropathic pain.

To induce allodynia, male Sprague-Dawley rats underwent chronic constriction injury (CCI) to the sciatic nerve as described by Bennett and Xie, Pain 1988; 33(1):87-107. The plantar surface of the hind paws was stimulated with von Frey filaments (Stoelting) to induce a withdrawal response by blinded personnel. The bending force of fiber required to induce a 50% withdrawal response was calculated following CCI surgery (pre-dosing baseline). N=5-6 allodynic rats received a single IP or oral administration of test compounds or vehicle. Two hours post-dosing, 50% paw withdrawal threshold was determined again by blinded testers using von Frey filaments. The 50% withdrawal threshold prior to CCI surgery, pre-dosing (10 days post-surgery), and 2 hours post-dosing are plotted.

For selected compounds multi-day studies were perfumed with pre-determined schedules for dosing and withdrawal threshold assessments. Results are shown in Table 3 below. The results indicate that multiple of the compounds evaluated are capable of attenuating mechanical allodynia and thus may be useful in the treatment of neuropathic pain.

FIG. 6 demonstrates the efficacy of AV1173 in the CCI model described above. As can be seen, upon oral dosing, AV1173 was capable of reversing allodynia and sustaining the efficacy overnight (24 hr time-points).

### Example 6

### Pharmacokinetic Evaluation

Pharmacokinetic parameters were determined for several of the compounds provided herein.

Three male Sprague-Dawley rats were dosed orally via gavage with 15 mg/kg of test compound. Serial blood samples were collected from the jugular vein at 5, 15, and 30 min, 1, 3, and 6 hours post dosing. Samples were processed for plasma via centrifugation and plasma samples were stored frozen prior to analysis via a sensitive and specific HPLC/MS/MS method. PK parameters were calculated using WinNonLin (Pharsight).

Oral pharmacokinetic values (Cₘₐₓ, AUCₗₐₛₜ, and T_{1/2}) are provided in Table 4 below. Three illustrative compounds, AV1173, AV1195, and AV1200, were found to possess enhanced plasma exposure following oral dosing in comparison to other compounds evaluated while still retaining their multi-target activities. The oral exposure of these compounds were enhanced several-fold, i.e, anywhere from 5-fold to 40-fold over the other compounds evaluated (e.g., AV1137, 1153, 1164, 1165, 1180, 1183, 1184, and 1198). Notably, both the Cₘₐₓ and AUC values for compound AV1200 were notably improved over those of the other compounds, including compound AV1173, also considered to possess an advantageous oral bioavailability.

TABLE 4

| | | IP CCI | PO CCI |
|---|---|---|---|
| | Oral PK | efficacy | efficacy |
| | (Cmax, | (Change in | (Change in |
| Compound | AUClast, T½) | grams) | grams) |
| SP600125 | | 0.5 | |
| Papaverine | | 1.35 | |
| AV1137 | 26/52/1.2 h | 3.26 | 0.94 (75 mg/kg) |
| AV1134 | | 0 | |
| AV1135 | | 0 | |
| AV1136 | | 0 | |
| AV1139 | | 0.21 | |
| AV1153 | 13/32/NA | 1.85 | |
| AV1154 | | 0 | |
| AV1155 | | 0.67 | |
| AV1156 | | 0.33 | |
| AV1158 | | 0.93 | |
| AV1159 | | 0 | |
| AV1164 | 17/12/N/A | 1.56 | |
| AV1165 | <5/<5/N/A | 0.44 | |
| AV1168 | | 0 | |
| AV1173 | 122/462/9.4 h | 2.5 | 1.6 (30 mg/kg) |
| AV1180 | 58/160/1.8 h | 0 | |
| AV1183 | 56/35/1.1 h | 0.3 | |
| AV1184 | <5/<10/N/A | | |
| AV1195 | 211/581/1.2 h | [0] | |
| AV1198 | [30/79/3.6 h] | [0] | |
| AV1200 | 420/1702/ | 0 (10 mg/kg); | 0 (30 mg/kg); |
| | 4.7 h | 1.8 (20 mg/kg) | 1.25 (60 mg/kg) |

The enhanced oral exposure for example compounds 1173 and 1200 may allow for eventual once or twice daily oral dosing in humans.

As can be seen from the foregoing illustrative examples, compounds have been prepared that possess activity against both phosphodiesterases (PDE 4 and 10) and JNK kinases. The discovered multi-target activity (phosphodiesterase and JNK kinase inhibition) is unique, and suggests that these compounds may be useful in treating multiple indications, e.g., neurodegenerative diseases such as Parkinson's and Alzheimer's, in addition to treating neuropathic pain.

Further, the compounds described are capable of regulating glial cell activation. The pathological role of glial cell activation has become apparent not only in neurodegenerative diseases and chronic pain states, but also in substance abuse and dependence (Hutchinson, M. R., Brain Behav Immun 2009 February; 23(2): 240-50), traumatic or ischemic injury (Hailer N P, Prog Neurobiol 84(3): 211-33, 2008), infection (Rock R B Clin Microbiol Rev 17(4): 942-64, 2004), and neoplasia (Krumbholz M, J Exp Med 201(2): 195-200, 2005; Sierra A, Lab Invest 77(4): 357-681997). Therefore the compounds described herein may have therapeutic benefit in a wide range of indications.

The invention(s) set forth herein has been described with respect to particular exemplified embodiments. However, the foregoing description is not intended to limit the invention to the exemplified embodiments, and the skilled artisan should recognize that variations can be made within the spirit and scope of the invention as described in the foregoing specification.

We claim:

1. A compound according to Formula I 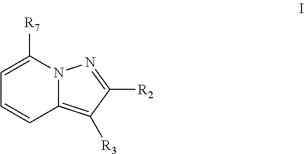
wherein
R₂ is selected from the group consisting of H, alkyl, alokoxyalkylene, cycloalkyl, phenyl and halophenyl;
R₃ is selected from the group consisting of 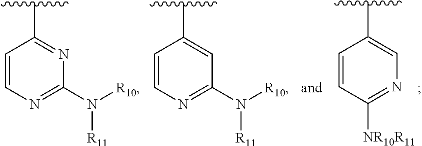
and
R₇ is selected from the group consisting H and methyl;
each of R₁₀ and R₁₁ are independently selected from the group consisting of H, substituted alkyl, cycloalkyl, S(O)₂R′ and aliphatic nitrogen heterocycles; and
wherein R′ is selected from the group consisting alkyl, aryl and heteroaryl.

2. The compound of claim 1, wherein —NR₁₀R₁₁ is selected from the group

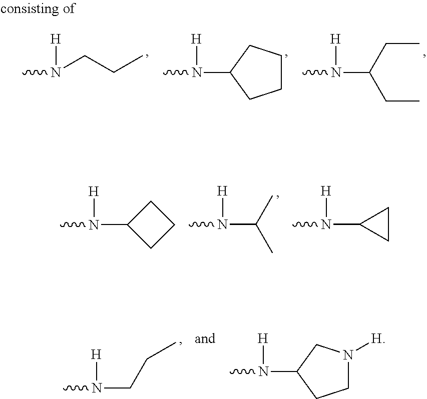

3. The compound of claim 1, wherein R₃ is 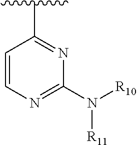
and R₁₀, and R₁₁ are each independently H.

4. The compound of claim 1, wherein R₃ is 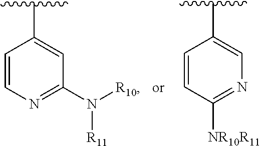
and R₁₀ and R₁₁ are each independently H.

5. The compound of claim 1, wherein when R′ is heteroaryl, R′ is a thiophene or a quinoline.

6. The compound of claim 1, wherein R′ is a phenyl or a methyl.

7. A compound that is selected from the following table:

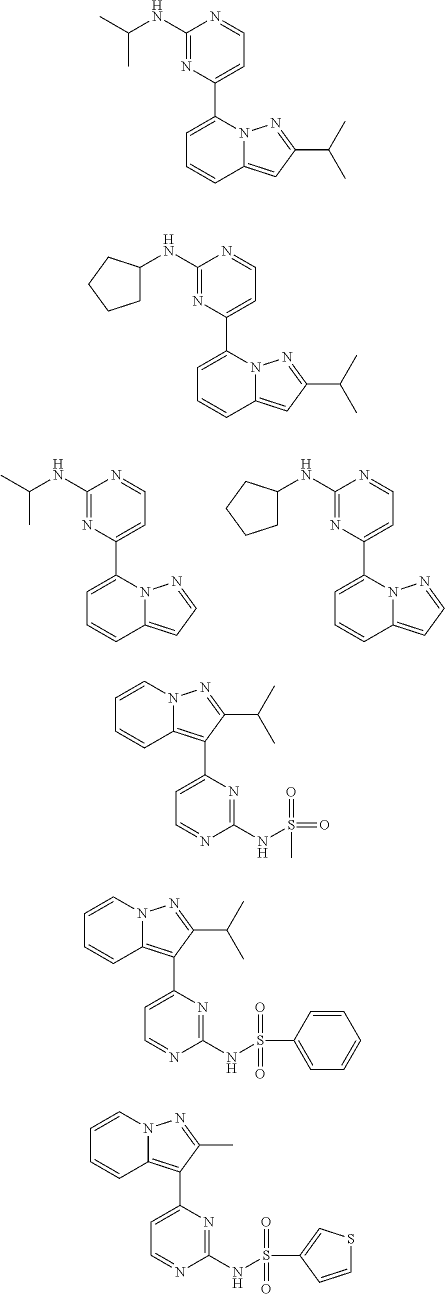

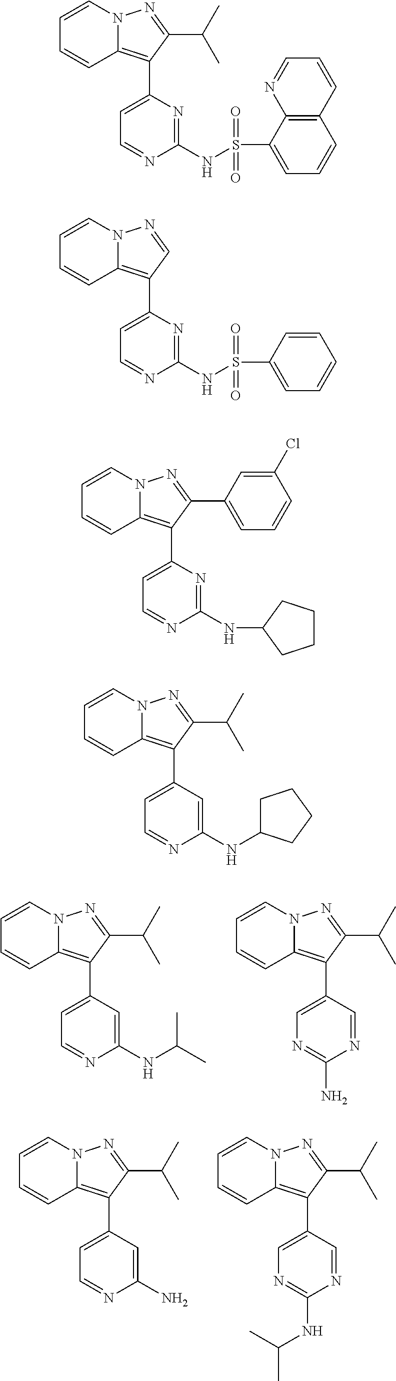

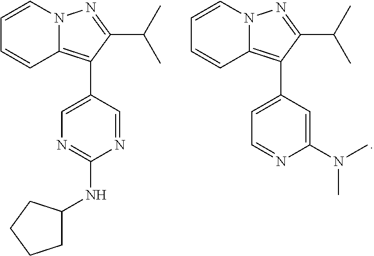

8. A pharmaceutical composition comprising (i) a compound that is selected from the following table: 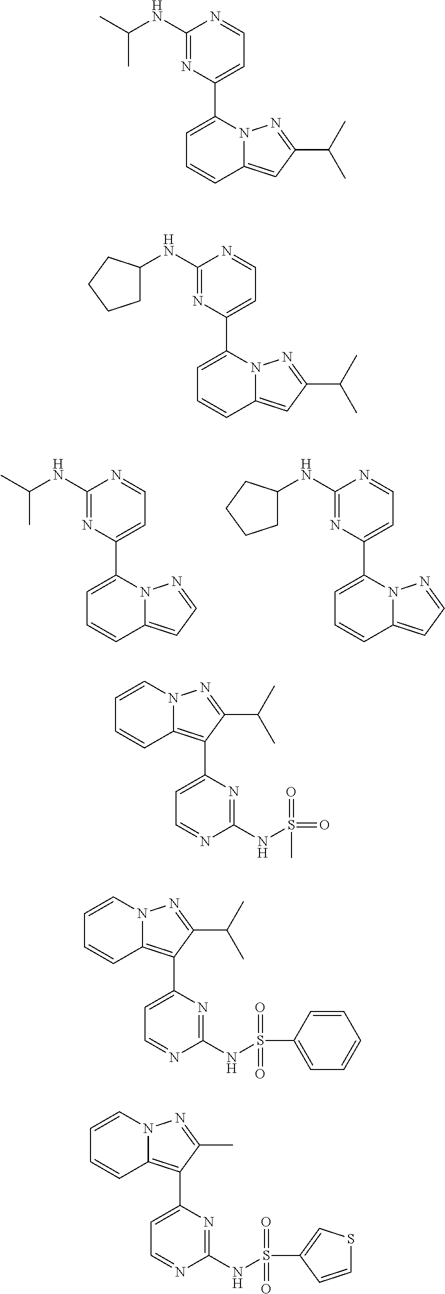 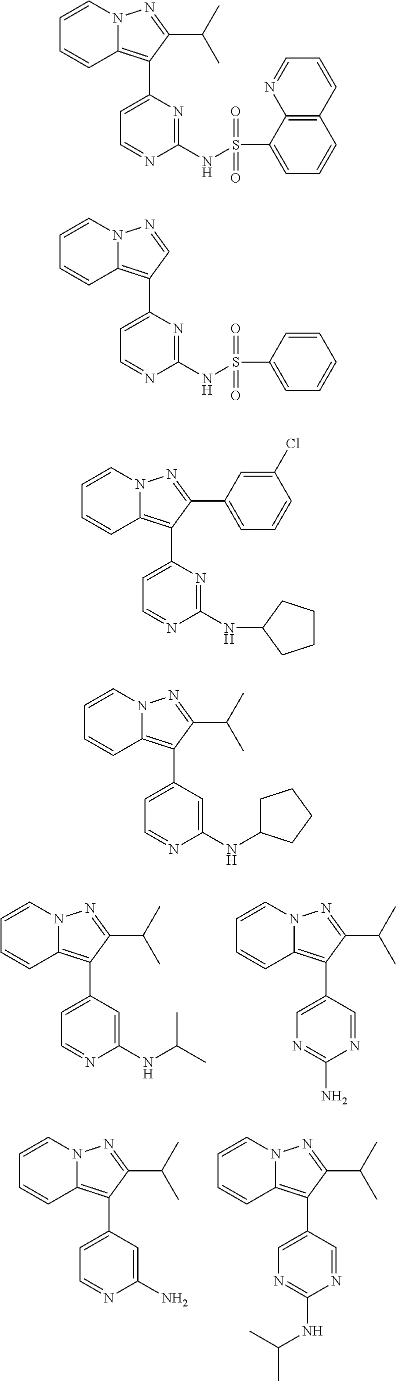 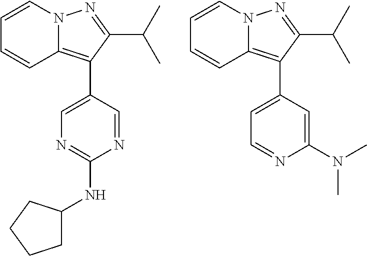
or pharmaceutically acceptable salts thereof; and
(ii) a pharmaceutically acceptable carrier.

9. The pharmaceutical composition according to claim 8, wherein the compound is 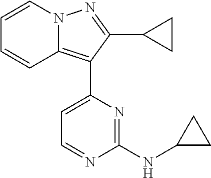
or its pharmaceutically acceptable salts.

10. A compound according to claim 1, wherein said compound inhibits the enzymes selected from the group consisting of phosphodiesterase (PDE) and c-Jun-N-terminal kinase (JNK).

11. The compound according to claim 7, wherein said compound inhibits at least one of PDE 4 or PDE 10.

12. The compound according to claim 11, wherein said compound inhibits at least one of JNK2 or JNK3.

13. A method for treating neuropathic pain, said method comprising administering a therapeutically effective amount of a compound according to claim 1, wherein said compound inhibits phosphodiesterase activity (PDE), c-Jun-N-terminal kinase (JNK) activity, or the activity of PDE and JNK, to treat or reduce neuropathic pain.
